(19) United States
(12) Patent Application Publication
(10) Pub. No.: US 2024/0122955 A1
(43) Pub. Date: Apr. 18, 2024

(54) ORAL DELIVERY

(71) Applicant: GUANGZHOU DAZHOU BIOMEDICINE LTD., Guangzhou (CN)

(72) Inventors: Steven DINH, Miami, FL (US); Huadong TANG, McLean, VA (US); Wenfei LIANG, Guangzhou (CN)

(21) Appl. No.: 18/084,897

(22) Filed: Dec. 20, 2022

(30) Foreign Application Priority Data
Dec. 29, 2021 (WO) PCT/CN2021/142542

(51) Int. Cl. A61K 31/7048; A61K 9/00; A61K 9/20; A61K 31/166; A61K 38/26

(52) U.S. Cl. CPC A61K 31/7048; A61K 9/0053; A61K 9/2095; A61K 31/166; A61K 38/26

(57) ABSTRACT

Provided herein are pharmaceutical compositions comprising therapeutic agent(s) and functional excipients that can enable oral absorption of small molecules and/or large molecules. Also provided herein are methods of preparing the pharmaceutical composition and methods of using the same for treating various diseases or disorders such as type-2 diabetes, obesity, NASH, renal disease.

### CROSS REFERENCE TO RELATED APPLICATIONS

This application claims priority to International Application No. PCT/CN2021/142542, filed Dec. 29, 2021, the entire contents of which are incorporated herein by reference.

### REFERENCE TO SEQUENCE LISTING SUBMITTED ELECTRONICALLY

The content of the electronically submitted sequence listing (Name: 6882_1010_Revised_ST26.xml, Size: 5 kilobytes, and Date of Creation: Jul. 31, 2023) is herein incorporated by reference in its entirety.

### BACKGROUND

### Field of the Invention

In various embodiments, the present invention generally relates to oral delivery of therapeutic agents, such as small molecule drugs and/or large molecule drugs, including but not limited to GLP-1 receptor agonists (GLP-1 RA) and/or SGLT2 inhibitors.

### Background Art

Delivery via oral route is one of the most preferred routes for drug administration. Oral route of administration has several advantages with better patient compliance, ease of administration and typically low cost of production, storage and distribution.

For large molecules, however, very few can be administered via the oral route for the following reasons: 1) pre-systemic degradation due to the acidity in the stomach, and enzymes in the gastro-intestinal (GI) tract; 2) low absorption across epithelial cells that line absorption surfaces such as those in the GI tract; and 3) post absorption degradation, such as the first pass metabolism.

For the combinations of large molecules and small molecules, there are more obstacles via oral delivery, because of the different physic-chemical, biological, drug dispositional, metabolic, etc., properties, etc. between large molecules and small molecules. Therefore, delivery of combinations of large molecules with small molecules adds another significant barrier, in addition to the delivery of large molecules alone.

In the field of treatments of metabolic diseases (such as diabetes, obesity, liver diseases, etc.), cardiovascular diseases, renal diseases, polypeptide, such as incretins (such as GLP-1 RA etc.), and small molecules such as SGLT2 inhibitors are important classes of therapeutics. However, these two types of classes of molecules, one is large and one is small, are extremely difficult to be able to be formulated into one oral dosage form. No successful commercial product of such combinations has been reported. The availability of these two types of molecules in one oral dosage form will not only brings in significant use convenience and compliance, compared to needle-based injection, but also adds synergistic medical benefits, such as enhanced glycemic control, body weight control, cardiovascular and renal benefits, etc., due to their different mechanisms of actions between large and small molecules.

### BRIEF SUMMARY

In various embodiments, the present disclosure relates to oral delivery of therapeutic agents, such as small molecule drugs (“small molecules”), optionally in combination with large molecule drugs (“large molecules”). In some particular embodiments, the present disclosure relates to combinations of large and small molecules such as those large molecules having high molecular weight or otherwise difficult to be absorbed through oral administration, such as polypeptides, etc., with small molecules with different mechanism of actions, in one oral dosage form, in other words, in a fixed-dose combination dosage form. The present disclosure is based, in part, on the discovery that small molecules such as SGLT-2 inhibitors can be formulated with functional excipients such as SNAC and sodium caprate for oral delivery and that the combination of certain fatty acids and oral absorption enhancers can achieve a synergistic effect in enhancing overall oral absorption of therapeutic agents, and in particular, on the unexpected discovery that the combinations of large and small molecules not only do not interfere with absorptions of each entities, but also enhance the absorption and improve the time-course pharmacokinetic profiles of small molecules.

In some embodiments, the present disclosure pertains to the use of one or more functional excipients for the preparation of oral formulations comprising small molecule(s) as a single agent or in combination with a large molecule or one or more other small molecules. In some embodiments, the present disclosure pertains to the use of mixtures of functional excipients combined with formulation method of preparation to enhance the gastrointestinal absorption of large molecules such as biologic therapeutics as a single agent or in combination with a small molecule or one or more other large molecules. The inventors believe that the disclosure herein provides compositions and methods that can transform approaches by which diseases are treated, cured and/or alleviated.

Small molecules as used herein are not particularly limited, and include for example SGLT-2 inhibitors, such as empagliflozin, canagliflozin, dapagliflozin, or ertugliflozin. For example, in some embodiments, the pharmaceutical composition herein can include an SGLT-2 inhibitor, optionally in combination with a large molecule described herein, such as an incretin, such as GLP-1 RA as described herein.

Large molecules as used herein are also not particularly limited, and include carbohydrates, peptides, proteins, enzymes, antibodies, drug conjugates, vaccines, nucleic acids and nucleic acid-based gene therapies. For example, in some embodiments, the pharmaceutical composition herein can include an incretin, including but not limited GLP-1 receptor agonist (“GLP-1 RA”, such as but not limited to semaglutide, liraglutide, exenatide), tirzepatide, PYY, oxyntomodulin, GLP-1, GLP-2, GIP (glucose-dependent insulinotropic polypeptides), Amylin or Amylin analogues, etc., as one therapeutic agent in combination with one or more other small molecule therapeutic agents that is useful for treating diabetes, obesity, cardiovascular, renal diseases, e.g., any of those known in the art. In some embodiments, the one or more other therapeutic agent can include (1) a SGLT-2 inhibitor, such as empagliflozin, canagliflozin, dapagliflozin, or ertugliflozin; (2) a DPP-4 inhibitor, such as sitagliptin, vildagliptin, saxagliptin, linagliptin, or alogliptin. In some embodiments, the one or more other therapeutic agent can also include (1) biguanides; (2) Thiazolidinediones; (3) DPP-4 inhibitors; (4) PYY; and (5) sulfonylureas.

In some embodiments, the pharmaceutical composition herein can include the GLP-1 receptor agonist as one therapeutic agent in combination with one or more other therapeutic agent that is useful for treating a neurological disease, such as Alzheimer's Disease. For example, in some embodiments, the pharmaceutical composition herein can include semaglutide as one therapeutic agent in combination with (1) Cholinesterase inhibitors (e.g., Aricept, Exelon, Razadyne); (2) Glutamate regulators (e.g., Namenda); and/or (3) Orexin receptor antagonist (e.g., Belsomra).

Functional excipients useful herein include but not limited to these molecules and their analogs: sodium 8-(2-hydroxybenzamido)octanoate (SNAC), 10-((2-hydroxybenzoyl)amino)decanoate sodium (SNAD), 8-(N-2-hydroxy-5-chlorobenzoyl)-amino-caprylates (5CNAC), sodium N-(4-chlorosalicyloyl)-4-aminobutyrate (4-CNAB), sodium N-[8-(2-hydroxy-4-methoxy)bensoyl]amino caprylate (4-MOAC), Bis-3,6(4-fumarylaminobutyl)-2,5-diketopiperazine. In addition, functional excipients useful herein include, for example, linear fatty acids and their salts with the number of carbons in the aliphatic chain ranging from 2 to 20.

In certain illustrative embodiments, the medication is administered using oral dosage forms that contain an active agent of incretin therapeutics (“incretins”) such as GLP-1 receptor agonists (GLP-1 RA), functional excipients such as mixtures of fatty acids and surfactants, and common excipients used in oral dosage forms such as tablets and capsules. Functional excipients useful herein include but not limited to sodium N-[8(-2-hydroxybenzoyl)amino]caprylate (SNAC), 8-(N-2-hydroxy-5-chlorobenzoyl)-amino-caprylic acid (5-CNAC), [Bis-3,6(4-fumarylaminobutyl)-2,5-diketopiperazine, salts of linear fatty acids, and combinations thereof. In other embodiments, the oral dosage forms can contain combination of active agents such as but not limited to GLP-1 RA and SGLT-2 inhibitors, GLP-1 RA and DPP4 inhibitors, and GLP-1 RA and insulin. Incretins useful for embodiments herein include but not limited GLP-1, GIP, GLP-1/GIP agonists. Incretins useful for embodiments herein also include GIP, GLP-1/GIP agonist in clinical trials, GLP-1 RA and GLP-1 analogues including but not limited to semaglutide, liraglutide, dulaglutide, lixisenatide, exenatide and others. SGLT2 inhibitors useful for embodiments herein include but not limited to empagliflozin, canagliflozin, dapagliflozin, ertugliflozin and others. DPP4 inhibitors useful for embodiments herein include but not limited to sitagliptin, vildagliptin, saxagliptin, linagliptin, alogliptin, and others. In yet other embodiments, the present disclosure pertains to methods for the preparation of oral dosage forms such as tablets and capsules. In further embodiments, mixtures of functional excipients and methods of preparation described herein can also be applied to other routes of administration.

Exemplary embodiments of the present disclosure are also shown in claims 1-47 as described herein.

It is to be understood that both the foregoing summary and the following detailed description are exemplary and explanatory only, and are not restrictive of the invention herein.

### BRIEF DESCRIPTION OF THE FIGURES

FIG. 1 shows a comparison of mean pharmacokinetics of semaglutide in plasma in a linear scale following oral administration of (1) formulations containing 10 mg semaglutide, 300 mg SNAC, and 150 mg sodium caprate; (2) formulations containing 10 mg semaglutide, 300 mg SNAC, and 150 mg sodium caprate and 10 mg empagliflozin. Error bars indicate the standard deviations of concentrations of semaglutide at specified times.

FIG. 2 shows a comparison of mean pharmacokinetics of empagliflozin in plasma in a linear scale following oral administration of (1) commercial product of empagliflozin 10 mg (Jardiance); (2) formulations containing 10 mg semaglutide, 300 mg SNAC, and 150 mg sodium caprate and 10 mg empagliflozin. Error bars indicate the standard deviations of concentrations of empagliflozin at specified times.

### DETAILED DESCRIPTION

The present disclosure generally relates to oral delivery of therapeutic agents. As detailed herein, the present inventors have discovered that small molecules such as SGLT-2 inhibitors can be formulated with functional excipients such as SNAC and sodium caprate for oral delivery. The present inventors also found that the small molecules and functional excipients can be combined with large molecules such as semaglutide to achieve desired plasma levels of both the small molecule and the large molecule following oral administration. As shown in the Examples section herein, a fixed-dose combination dosage form comprising empagliflozin and semaglutide, with excipients including SNAC and sodium caprate can be prepared and can achieve a comparable or better pharmacokinetic profile compared to comparative formulations. This unexpected discovery supports that the combinations of large and small molecules in one dosage form can be formulated to achieve a comparable or better overall pharmacokinetic profile: it is now found that the large molecule and small molecule do not interfere with absorptions of each entity, and in the tested formulation, the small molecule, empagliflozin, even achieved enhanced absorption and/or improved time-course pharmacokinetic profiles, such as increased exposure (AUC), lower the maximal of small molecule, and lower maximal concentration (Cmax).

In a broad aspect, the present disclosure provides a pharmaceutical composition comprising a small molecule drug (e.g., any of those described herein) and optionally a large molecule drug, and one or more, particularly, two or more, functional excipients (e.g., any of those described herein). Unless otherwise contrary from context, functional excipients as used herein refer to those excipients that can enhance the oral bioavailability of at least one therapeutic agent of the pharmaceutical compositions herein, such as a large molecule drug. For example, in some embodiments, the functional excipients refer to those that can enable the oral absorption for a large and/or small molecule drug following oral administration. Typically, large molecule drugs refer to those having a molecular weight of more than 2,000 Daltons, more than 3,000 Daltons, more than 10,000 Daltons, or more than 100,000 Daltons, etc. Typically, small molecule drugs refer to those having a molecular weight of less than 2000 Daltons, such as less than 1000 Daltons or less than 500 Daltons.

Typically, the pharmaceutical composition comprises two or more functional excipients that can synergistically enhance the oral absorption of the therapeutic agent(s) herein. In some embodiments, the one or more functional excipients include an aliphatic acid of Formula I: RCOOH, wherein R represents an aliphatic group, or a pharmaceutically acceptable salt thereof. In some embodiments, the one or more functional excipients include a compound of Formula II:

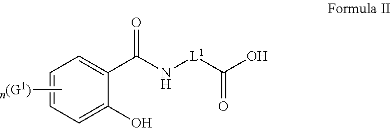

or a pharmaceutically acceptable salt thereof, wherein:
n is an integer selected from 0, 1, 2, 3, or 4;
G¹ at each occurrence is independently OH, NH₂, NH(C₁₋₄ alkyl), N(C₁₋₄ alkyl)(C₁₋₄ alkyl), halogen (e.g., Cl), C₁₋₄ alkyl, or C₁₋₄ alkoxy (e.g., OCH₃); and
L¹ is a substituted or unsubstituted C₂-C₁₆ alkylene, or substituted or unsubstituted C₂-C₁₆ alkenylene. Typically, the pharmaceutical composition is formulated for oral administration. Preferably, a therapeutically effective plasma concentration of the small molecule drug and optionally the large molecule drug can be achieved following oral administration of the pharmaceutical composition herein.

In some embodiments, the present disclosure provides a pharmaceutical composition comprising (a) a small molecule drug, such as an SGLT-2 inhibitor (e.g., any of those described herein), and one or more ingredients selected from (b) an aliphatic acid of Formula I: RCOOH, wherein R represents an aliphatic group, or a pharmaceutically acceptable salt thereof, and (c) a compound of Formula II:

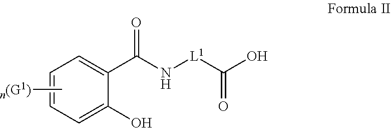

or a pharmaceutically acceptable salt thereof, wherein:
n is an integer selected from 0, 1, 2, 3, or 4;
G¹ at each occurrence is independently OH, NH₂, NH(C₁₋₄ alkyl), N(C₁₋₄ alkyl)(C₁₋₄ alkyl), halogen (e.g., Cl), C₁₋₄ alkyl, or C₁₋₄ alkoxy (e.g., OCH₃); and
L¹ is a substituted or unsubstituted C₂-C₁₆ alkylene, or substituted or unsubstituted C₂-C₁₆ alkenylene.

Therapeutic Agents

The pharmaceutical composition herein typically comprises a small molecule drug optionally in combination with a large molecule drug in one dosage form, such as a fixed-dose combination dosage form. In some preferred embodiments, the pharmaceutical composition herein comprises an SGLT-2 inhibitor. In some preferred embodiments, the pharmaceutical composition can further comprise an incretin.

For example, in some embodiments, the pharmaceutical composition comprises (a) an SGLT-2 inhibitor (e.g., any of those described herein); and (b) the aliphatic acid of Formula I (e.g., any of those described herein), or a pharmaceutically acceptable salt thereof. In some embodiments, the pharmaceutical composition comprises (a) an SGLT-2 inhibitor (e.g., any of those described herein); and (c) the compound of Formula II (e.g., any of those described herein), or a pharmaceutically acceptable salt thereof. In some embodiments, the pharmaceutical composition comprises (a) the SGLT-2 inhibitor, (b) the aliphatic acid of Formula I (e.g., any of those described herein), or a pharmaceutically acceptable salt thereof; and (c) the compound of Formula II (e.g., any of those described herein), or a pharmaceutically acceptable salt thereof. In some embodiments, the pharmaceutical composition further comprises an incretin (e.g., a GLP-1 RA described herein). The pharmaceutical composition is typically formulated for oral administration. In some embodiments, upon oral administration to a human subject in need thereof, the pharmaceutical composition can deliver a therapeutically effective amount of the SGLT-2 inhibitor to the human subject. In some embodiments, upon oral administration to a human subject in need thereof, the applicable pharmaceutical composition can deliver a therapeutically effective amount of the SGLT-2 inhibitor and the incretin (e.g., a GLP-1 RA described herein) to the human subject.

SGLT-2 inhibitors useful for the pharmaceutical compositions described herein are not particularly limited. SGLT2 inhibitors (sodium-glucose co-transporter 2) represent a novel class of agents that can be used for the treatment or improvement in glycemic control in patients with type 2 diabetes. Useful SGLT-2 inhibitors include empagliflozin, canagliflozin, dapagliflozin, or ertugliflozin. Other useful SGLT-2 inhibitors include those known in the art such as those described in U.S. Pat. Nos. 6,515,117, 7,579,449, 7,919,598, 8,202,984, 8,080,580, 10,617,668, International PCT Application Publication Nos. WO 01/27128, WO 03/099836, WO 2005/092877, WO 2005/012326, WO 2006/034489, WO 2006/064033, WO 2006/117359, WO 2006/117360, WO 2007/025943, WO 2007/028814, WO 2007/031548, WO 2007/093610, WO 2007/128749, WO 2008/049923, WO 2008/055870, and WO 2008/055940. In some preferred embodiments, the pharmaceutical composition herein comprises empagliflozin.

Incretins useful for the pharmaceutical compositions described herein are also not particularly limited. Useful incretins include any of those known in the art such as GLP-1 RAs such as but not limited to semaglutide, liraglutide, exenatide, tirzepatide, PYY, oxyntomodulin, GLP-1, GLP-2, GIP (glucose-dependent insulinotropic polypeptides), Amylin or Amylin analogues, etc.

For example, in some preferred embodiments, the pharmaceutical composition can include a Glucagon-Like Peptide-1 (GLP-1) receptor agonist, e.g., any of those described herein, or any of those described in U.S. Pat. Nos. 10,960,052, 8,129,343, 8,536,122, 9,278,123, 10,086,047, 10,278,923, and 10,933,120, the entire contents of each of which are herein incorporated by reference. In some preferred embodiments, the pharmaceutical composition can include semaglutide, liraglutide, dulaglutide, lixisenatide, or exenatide. Other incretins and peptides can include, but not limited to, PYY and PYY analogues; GLP-1/GIP receptor due agonists such as, but not limited to Tirzepatide, CT-388, SCO-094, etc.; GLP-1/GCGR receptor due agonists, such as, but not limited to efinopegdutide, IB 1362, etc.

In some embodiments, the pharmaceutical composition can be used for the treatment of metabolic, cardiovascular and renal diseases.

In some embodiments, the pharmaceutical composition can further include one or more other small molecule therapeutic agents that is useful for treating diabetes, obesity, cardiovascular, renal diseases, e.g., (i) a DPP-4 inhibitor, such as sitagliptin, vildagliptin, saxagliptin, linagliptin, or alogliptin. In some embodiments, the pharmaceutical composition can also include (ii) biguanides; (iii) Thiazolidinediones; (iv) DPP-4 inhibitors; (v) PYY; and/or (vi) sulfonylureas. In some embodiments, the one or more other therapeutic agent can include one or more selected from the following: Biguanides, Sulfonylureas and meglitinides, Thiazolidinediones, Alpha-glucosidase inhibitors, other Glucagon like peptide-1 (GLP-1) receptor agonists, Dipeptidyl peptidase 4 (DPP4) inhibitors, Amylin analogue, Sodium-glucose cotransporter 2 (SGLT-2) inhibitors, Dopamine agonists, and Bile acid sequestrants. In some embodiments, the one or more other therapeutic agent can include one or more selected from the following: Metformin, Glipizide, Gliclazide, Glyburide, Glimepiride, Nateglinide, Repaglinide, Pioglitazone, Rosiglitazone, Acarbose, Miglitol, Voglibose, Exenatide, Liraglutide, Lixisenatide, Dulaglutide, Albiglutide, Sitagliptin, Vildagliptin, Saxagliptin, Linagliptin, Gemigliptin, Anagliptin, Teneligliptin, Alogliptin, Trelagliptin, Omarigliptin, Evogliptin, Gosogliptin, Pramlintide, Canagliflozin, Dapagliflozin, Empagliflozin, Ipragliflozin, Bromocriptine, and Colesevelam.

In some embodiments, the pharmaceutical composition can be used for the treatment of neurological diseases, such as Alzheimer's Disease. For example, in some embodiments, the pharmaceutical composition herein can include semaglutide as one therapeutic agent in combination with (1) Cholinesterase inhibitors (e.g., Aricept, Exelon, Razadyne); (2) Glutamate regulators (e.g., Namenda); and/or (3) Orexin receptor antagonist (e.g., Belsomra).

In more preferred embodiments, the pharmaceutical composition herein can include semaglutide. Semaglutide as used herein is not limited to any particular forms. For example, in some embodiments, semaglutide can be in the form of a pharmaceutically acceptable salt, such as a sodium salt. Semaglutide is marketed in the United States under several brandnames, including the oral Rybelsus® tablet formulation. See Rybelsus Prescribing Information approved by the U.S. Food and Drug Administration, 2021 version, the content of which is herein incorporated by reference in its entirety. As described therein, the peptide backbone of semaglutide is produced by yeast fermentation. The main protraction mechanism of semaglutide is albumin binding, facilitated by modification of position 26 lysine with a hydrophilic spacer and a C18 fatty di-acid. Furthermore, semaglutide is modified in position 8 to provide stabilization against degradation by the enzyme dipeptidyl-peptidase 4 (DPP-4). A minor modification was made in position 34 to ensure the attachment of only one fatty di-acid. The molecular formula is C187H291N45059 and the molecular weight is 4113.58 g/mol. The structure is shown below:

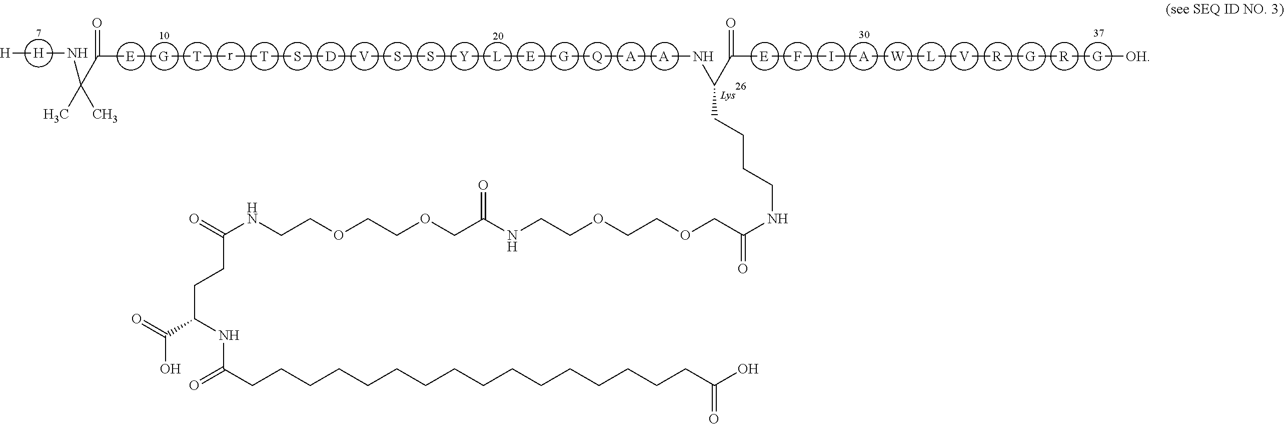

In more preferred embodiments, the pharmaceutical composition herein can include empagliflozin. Empagliflozin as used herein is not limited to any particular forms. Empagliflozin is marketed in the United States under Jardiance. Molecular weight of empagliflozin is 450.91 g/mol. Formula is C23H27C107. The structure of empagliflozin is shown below:

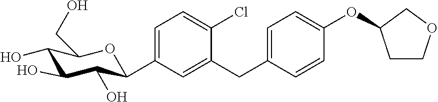

Methods of synthesis of empagliflozin are described in the literature, for example WO 06/120208 and WO 2011/039108. It is to be understood that the definition of empagliflozin also comprises its hydrates, solvates and polymorphic forms thereof, and prodrugs thereof. An advantageous crystalline form of empagliflozin is described in WO 2006/117359 and WO 2011/039107 which hereby are incorporated herein in their entirety. This crystalline form possesses good solubility properties which enables a good bioavailability of the SGLT2 inhibitor. Furthermore, the crystalline form is physico-chemically stable and thus provides a good shelf-life stability of the pharmaceutical composition. Preferred pharmaceutical compositions, such as solid formulations for oral administration, for example tablets, are described in WO 2010/092126, which is incorporated herein in its entirety.

### Aliphatic Acid of Formula I

Typically, the pharmaceutical composition herein comprises an aliphatic acid of Formula I: RCOOH, wherein R represents an aliphatic group, or a pharmaceutically acceptable salt thereof.

Useful aliphatic acids are not particularly limited. For example, in some embodiments, the aliphatic acid has a Formula I: RCOOH, wherein R represents an alkyl group having 1-30 carbon atoms. The alkyl group can be a linear or branched chain alkyl group. For example, in some embodiments, R in Formula I can be —(CH₂)₁₋₁₈CH₃. In some embodiments, R in Formula I can be an alkyl group having 3-20 carbon atoms. In some embodiments, R in Formula I can be an alkyl group having 5-16 carbon atoms. In some embodiments, the aliphatic acid of Formula I is a linear aliphatic acid having 2 to 20 carbon atoms, such as caprylic acid, capric acid, or lauric acid. In any of the embodiments described herein, unless otherwise specified or contrary from context, the aliphatic acid of Formula I can be capric acid.

The aliphatic acid of Formula I can be present in the pharmaceutical composition herein as a free acid or any pharmaceutically acceptable salt thereof, such as an alkali or alkaline salt thereof, for example, a sodium or potassium salt. In some preferred embodiments, the pharmaceutical composition herein comprises sodium caprate.

### Compound of Formula II

Typically, the pharmaceutical composition herein comprises a compound of Formula II:

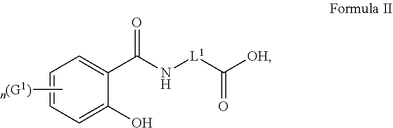

or a pharmaceutically acceptable salt thereof, wherein the variables are defined herein.

In some embodiments, the compound of Formula II can have no G¹ substituents on the phenyl ring, i.e., n is 0.

In some embodiments, the compound of Formula II can have one G¹ substituted on the phenyl ring, i.e., n is 1. In some embodiments, in Formula II, n is 1, and G¹ is a halogen, C₁₋₄ alkyl, or C₁-4 alkoxy. In some embodiments, in Formula II, n is 1, and G¹ is C₁. In some embodiments, in Formula II, n is 1, and G¹ is OCH₃.

L¹ in Formula II is typically a substituted or unsubstituted C₂-C₁₆ alkylene. For example, in some embodiments, L¹ is an unsubstituted C₃-C₁₅ alkylene. In some embodiments, L¹ is an unsubstituted C₅-C₁₃ alkylene. The alkyelene can be a straight-chained or a branched alkyelene. For example, in some embodiments, L¹ is an unsubstituted, straight-chained C₅-C₉ alkylene.

In some preferred embodiments, the compound of Formula II can be

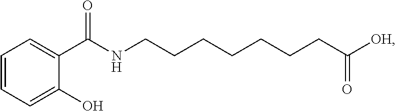 [SMILES: O=C(O)CCCCCCCNC(=O)c1ccccc1O]

which has a chemical name of 8-(2-hydroxybenzamido)octanoic acid (ChemDraw Software, version 20.0). In preferred embodiments, the pharmaceutical composition herein comprises a salt (preferably sodium salt) of 8-(2-hydroxybenzamido)octanoic acid, which can be prepared using the method described in e.g. WO96/030036, WO00/046182, WO01/092206 or WO2008/028859. The salt of 8-(2-hydroxybenzamido)octanoic acid (alternatively known as N-(8-(2-hydroxybenzoyl)amino)caprylic acid) may be crystalline and/or amorphous. In some embodiments the delivery agent comprises the anhydrate, monohydrate, dihydrate, trihydrate, a solvate or one third of a hydrate of the salt of N-(8-(2-hydroxybenzoyl)amino) caprylic acid as well as combinations thereof. In some embodiments, the pharmaceutical composition herein comprises a salt of N-(8-(2-hydroxybenzoyl)amino)caprylic acid as described in WO2007/121318.

In more preferred embodiments, the pharmaceutical composition comprises sodium N-(8-(2-hydroxybenzoyl)amino) caprylate (referred to as “SNAC” herein).

In some preferred embodiments, the compound of Formula II can be

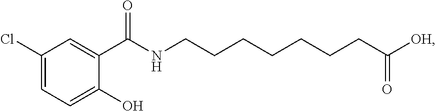 [SMILES: O=C(O)CCCCCCCNC(=O)c1cc(Cl)ccc1O]

which has a chemical name of 8-(5-chloro-2-hydroxybenzamido)octanoic acid (ChemDraw Software, version 20.0). In some embodiments, the pharmaceutical composition herein comprises a salt of 8-(5-chloro-2-hydroxybenzamido)octanoic acid.

In some embodiments, the compound of Formula II can be

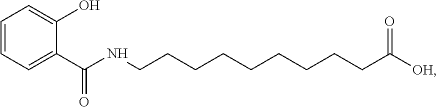 [SMILES: O=C(O)CCCCCCCCCNC(=O)c1ccccc1O]

which has a chemical name 10-((2-hydroxybenzoyl)amino)decanoic acid. In some embodiments, the pharmaceutical composition herein comprises a salt of 10-((2-hydroxybenzoyl)amino)decanoic acid, such as sodium 10-((2-hydroxybenzoyl)amino)decanoate.

In some embodiments, the compound of Formula II can be

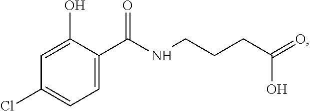 [SMILES: O=C(O)CCCNC(=O)c1ccc(Cl)cc1O]

which has a chemical name N-(4-chlorosalicyloyl)-4-aminobutyric acid. In some embodiments, the pharmaceutical composition herein comprises a salt of N-(4-chlorosalicyloyl)-4-aminobutyric acid, such as sodium N-(4-chlorosalicyloyl)-4-aminobutyrate.

In some embodiments, the compound of Formula II can be

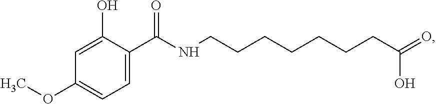 [SMILES: COc1ccc(C(=O)NCCCCCCCC(=O)O)c(O)c1]

which has a chemical name N48-(2-hydroxy-4-methoxy)benzoyl]amino caprylic acid. In some embodiments, the pharmaceutical composition herein comprises a salt of N-[8-(2-hydroxy-4-methoxy)benzoyl]amino caprylic acid, such as sodium N-[8-(2-hydroxy-4-methoxy)benzoyl]amino caprylate.

The combinations of therapeutic agent(s) herein, aliphatic acid of Formula I and the compound of Formula II are not particularly limited.

In some preferred embodiments, the pharmaceutical composition herein comprises (a) an SGLT2 inhibitor (e.g., any of those described herein, such as empagliflozin); (b) capric acid or a pharmaceutically acceptable salt thereof; and (c) the compound of Formula II or a pharmaceutically acceptable salt thereof.

In some preferred embodiments, the pharmaceutical composition herein comprises (a) an SGLT2 inhibitor (e.g., any of those described herein, such as empagliflozin); (b) a linear aliphatic acid having 2 to 20 carbon atoms, such as caprylic acid, capric acid, or lauric acid or a pharmaceutically acceptable salt thereof; and (c) SNAC.

In some preferred embodiments, the pharmaceutical composition herein comprises (a) an SGLT2 inhibitor (e.g., any of those described herein, such as empagliflozin); (b) capric acid or a pharmaceutically acceptable salt thereof; and (c) 8-(2-hydroxybenzamido)octanoic acid or a pharmaceutically acceptable salt thereof.

In some preferred embodiments, the pharmaceutical composition herein comprises (a) an SGLT2 inhibitor (e.g., any of those described herein, such as empagliflozin); (b) sodium caprate; and (c) SNAC.

In some preferred embodiments, the pharmaceutical composition herein comprises (a) empagliflozin; (b) sodium caprate; and (c) SNAC.

In some preferred embodiments, the pharmaceutical composition herein comprises (a) an SGLT2 inhibitor (e.g., any of those described herein, such as empagliflozin) and a large molecule drug (e.g., any of those described herein); (b) capric acid or a pharmaceutically acceptable salt thereof; and (c) the compound of Formula II or a pharmaceutically acceptable salt thereof.

In some preferred embodiments, the pharmaceutical composition herein comprises (a) an SGLT2 inhibitor (e.g., any of those described herein, such as empagliflozin) and a large molecule drug (e.g., any of those described herein); (b) a linear aliphatic acid having 2 to 20 carbon atoms, such as caprylic acid, capric acid, or lauric acid or a pharmaceutically acceptable salt thereof; and (c) SNAC.

In some preferred embodiments, the pharmaceutical composition herein comprises (a) an SGLT2 inhibitor (e.g., any of those described herein, such as empagliflozin) and a large molecule drug (e.g., any of those described herein); (b) capric acid or a pharmaceutically acceptable salt thereof; and (c) 8-(2-hydroxybenzamido)octanoic acid or a pharmaceutically acceptable salt thereof.

In some preferred embodiments, the pharmaceutical composition herein comprises (a) an SGLT2 inhibitor (e.g., any of those described herein, such as empagliflozin) and a large molecule drug (e.g., any of those described herein); (b) sodium caprate; and (c) SNAC.

In some preferred embodiments, the pharmaceutical composition herein comprises (a) empagliflozin and a large molecule drug (e.g., any of those described herein); (b) sodium caprate; and (c) SNAC.

In some preferred embodiments, the pharmaceutical composition herein comprises (a) an SGLT2 inhibitor (e.g., any of those described herein, such as empagliflozin) and an incretin (e.g., any of those described herein, such as semaglutide); (b) capric acid or a pharmaceutically acceptable salt thereof; and (c) the compound of Formula II or a pharmaceutically acceptable salt thereof.

In some preferred embodiments, the pharmaceutical composition herein comprises (a) an SGLT2 inhibitor (e.g., any of those described herein, such as empagliflozin) and an incretin (e.g., any of those described herein, such as semaglutide); (b) a linear aliphatic acid having 2 to 20 carbon atoms, such as caprylic acid, capric acid, or lauric acid or a pharmaceutically acceptable salt thereof; and (c) SNAC.

In some preferred embodiments, the pharmaceutical composition herein comprises (a) an SGLT2 inhibitor (e.g., any of those described herein, such as empagliflozin) and an incretin (e.g., any of those described herein, such as semaglutide); (b) capric acid or a pharmaceutically acceptable salt thereof; and (c) 8-(2-hydroxybenzamido)octanoic acid or a pharmaceutically acceptable salt thereof.

In some preferred embodiments, the pharmaceutical composition herein comprises (a) an SGLT2 inhibitor (e.g., any of those described herein, such as empagliflozin) and an incretin (e.g., any of those described herein, such as semaglutide); (b) sodium caprate; and (c) SNAC.

In some preferred embodiments, the pharmaceutical composition herein comprises (a) semaglutide and empagliflozin; (b) sodium caprate; and (c) SNAC.

Typically, in the pharmaceutical compositions herein, as applicable, the weight ratio of (b) the aliphatic acid of Formula I or pharmaceutically acceptable salt thereof to (c) the compound of Formula II or pharmaceutically acceptable salt thereof, (b)/(c), ranges from about 20:1 to about 1:20, such as 5:1 to 1:5, e.g., about 1:2.

The amounts of the therapeutic agent(s) herein, the aliphatic acid of Formula I or pharmaceutically acceptable salt thereof, and the compound of Formula II or pharmaceutically acceptable salt thereof, in the applicable pharmaceutical compositions herein are not particularly limited.

For example, in some embodiments, the pharmaceutical composition comprises the aliphatic acid of Formula I or pharmaceutically acceptable salt thereof in an amount of about 50 mg to about 300 mg per unit dose, such as about 50 mg, about 100 mg, about 150 mg, about 200 mg, about 300 mg, or any range between the recited value, per unit dose. As used herein, unless otherwise specified or obviously contrary from context, the weight of the aliphatic acid of Formula I or pharmaceutically acceptable salt thereof should be understood as the equivalent weight expressed as the weight of the free acid. However, when referring to the amount of sodium caprate specifically, the amount should be understood as the weight of the sodium salt itself, not the corresponding equivalent weight of capric acid. In some embodiments, the pharmaceutical composition comprises the aliphatic acid of Formula I or pharmaceutically acceptable salt thereof in an amount of at least 0.6 mmol (millimole), such as selected from the group consisting of at least 0.65 mmol, at least 0.7 mmol, at least 0.75 mmol, at least 0.8 mmol, at least 0.8 mmol, at least 0.9 mmol, at least 0.95 mmol and at least 1 mmol, per unit dose. In some embodiments, the pharmaceutical composition comprises the aliphatic acid of Formula I or pharmaceutically acceptable salt thereof in an amount of 0.6 mmol to 2 mmol, such as 0.8 mmol to 1.3 mmol, 0.9 mmol to 1.1 mmol, such as 0.95 mmol, 1.0 mmol, etc., per unit dose.

In some embodiments, the pharmaceutical composition comprises the compound of Formula II or pharmaceutically acceptable salt thereof in an amount of about 200 mg to about 400 mg per unit dose, such as about 200 mg, about 250 mg, about 300 mg, about 350 mg, about 400 mg, or any range between the recited value, per unit dose. As used herein, unless otherwise specified or obviously contrary from context, the weight of the compound of Formula II or pharmaceutically acceptable salt thereof should be understood as equivalent weight expressed as the weight of the compound of Formula II. However, when referring to the amount of SNAC specifically, the amount should be understood as the weight of the sodium salt itself, not the corresponding equivalent weight of the acid. In some embodiments, the pharmaceutical composition comprises the compound of Formula II or pharmaceutically acceptable salt thereof in an amount of at least 0.6 mmol, such as selected from the group consisting of at least 0.65 mmol, at least 0.7 mmol, at least 0.75 mmol, at least 0.8 mmol, at least 0.8 mmol, at least 0.9 mmol, at least 0.95 mmol and at least 1 mmol, per unit dose. In some embodiments, the pharmaceutical composition comprises the compound of Formula II or pharmaceutically acceptable salt thereof in an amount of 0.6 mmol to 2 mmol, such as 0.8 mmol to 1.3 mmol, or 0.9 mmol to 1.1 mmol, such as 1 mmol, per unit dose.

Typically, the pharmaceutical composition comprises a synergistic combination of (b) the aliphatic acid of Formula I or pharmaceutically acceptable salt thereof and (c) the compound of Formula II or pharmaceutically acceptable salt thereof, for achieving enhanced oral delivery of the SGLT-2 inhibitor and/or the optional large molecule drug, such as the incretin described herein.

In some specific embodiments, the pharmaceutical composition comprises (a) one large molecule drug and one small molecule drug (e.g., any of those described herein); (b) sodium caprate in an amount of about 50 mg to about 300 mg (e.g., about 100 mg, about 150 mg, about 200 mg, or any range between the recited values); and (c) SNAC in an amount of about 200 mg to about 400 mg (e.g., about 200 mg, about 300 mg, about 400 mg, or any range between the recited values), per unit dose. The amount of the large molecule drug is not particularly limited, for example, typically, the large molecule drug can be in an amount of about 1 mg to about 200 mg (e.g., about 1 mg, about 10 mg, about 20 mg, or any range between the recited values), per unit dose. The amount of the small molecule drug is also not particularly limited, for example, typically, the therapeutic agents can be in an amount of about 1 mg to about 1000 mg (e.g., about 1 mg, about 10 mg, about 20 mg, about 100 mg, about 500 mg, or any range between the recited values), per unit dose.

In some specific embodiments, the pharmaceutical composition comprises (a) an incretin (e.g., any of those described herein) and an SGLT2 inhibitor (e.g., any of those described herein); (b) sodium caprate in an amount of about 50 mg to about 300 mg (e.g., about 100 mg, about 150 mg, about 200 mg, or any range between the recited values); and (c) SNAC in an amount of about 200 mg to about 400 mg (e.g., about 200 mg, about 300 mg, about 400 mg, or any range between the recited values), per unit dose. The amount of the incretin is not particularly limited, for example, typically, the incretin can be in an amount of about 1 mg to about 200 mg (e.g., about 10 mg, about 50 mg, about 100 mg, or any range between the recited values), per unit dose. The amount of the SGLT2 inhibitor is not particularly limited, for example, typically, the SGLT2 inhibitor can be in an amount of about 1 mg to about 200 mg (e.g., about 1 mg, about 10 mg, about 20 mg, or any range between the recited values), per unit dose.

In some specific embodiments, the pharmaceutical composition comprises (a) a GLP-1 receptor agonist (e.g., any of those described herein) and an SGLT2 inhibitor (e.g., any of those described herein); (b) sodium caprate in an amount of about 50 mg to about 300 mg (e.g., about 100 mg, about 150 mg, about 200 mg, or any range between the recited values); and (c) SNAC in an amount of about 200 mg to about 400 mg (e.g., about 200 mg, about 300 mg, about 400 mg, or any range between the recited values), per unit dose. The amount of the GLP-1 receptor agonist is not particularly limited, for example, typically, the GLP-1 receptor agonist can be in an amount of about 1 mg to about 200 mg (e.g., about 10 mg, about 50 mg, about 100 mg, or any range between the recited values), per unit dose. The amount of the SGLT2 inhibitor is not particularly limited, for example, typically, the SGLT2 inhibitor can be in an amount of about 1 mg to about 200 mg (e.g., about 1 mg, about 10 mg, about 20 mg, or any range between the recited values), per unit dose.

In some specific embodiments, the combinations comprise (a) semaglutide and empagliflozin, (b) sodium caprate in an amount of about 50 mg to about 300 mg (e.g., about 100 mg, about 150 mg, about 200 mg, or any range between the recited values); and (c) SNAC in an amount of about 200 mg to about 400 mg (e.g., about 200 mg, about 300 mg, about 400 mg, or any range between the recited values), per unit dose. The amount of semaglutide is not particularly limited, for example, typically, the semaglutide can be in an amount of about 1 mg to about 200 mg (e.g., about 3 mg, about 10 mg, about 50 mg, about 100 mg, or any range between the recited values), per unit dose. The amount of empagliflozin is not particularly limited, for example, typically, the empagliflozin can be in an amount of about 1 mg to about 100 mg (e.g., about 5 mg, about 10 mg, about 20 mg, or any range between the recited values), per unit dose.

In any of the embodiments described herein, unless otherwise specified or contrary from context, the pharmaceutical composition herein comprising both the small molecule drug and the large molecule drug can be in a fixed-dose combination dosage form, in other words, the small molecule drug and the large molecule drug can be present in a single dosage form. For example, in some embodiments, the pharmaceutical composition herein can be a fixed-dose combination of empagliflozin and semaglutide in a single dosage form. For example, in some embodiments, each dosage unit of the single dosage form can include (a) semaglutide in an amount of about 1 mg to about 200 mg (e.g., about 3 mg, about 10 mg, about 50 mg, about 100 mg, or any range between the recited values) and empagliflozin in an amount of about 1 mg to about 100 mg (e.g., about 5 mg, about 10 mg, about 20 mg, or any range between the recited values); (b) sodium caprate in an amount of about 50 mg to about 300 mg (e.g., about 100 mg, about 150 mg, about 200 mg, or any range between the recited values); and (c) SNAC in an amount of about 200 mg to about 400 mg (e.g., about 200 mg, about 300 mg, about 400 mg, or any range between the recited values). In some embodiments, two or more dosage units (e.g., 2, 3, 4, 5, or 6 dosage units) of the single dosage form in combination can include (a) semaglutide in an amount of about 1 mg to about 200 mg (e.g., about 3 mg, about 10 mg, about 50 mg, about 100 mg, or any range between the recited values) and empagliflozin in an amount of about 1 mg to about 100 mg (e.g., about 5 mg, about 10 mg, about 20 mg, or any range between the recited values); (b) sodium caprate in an amount of about 50 mg to about 300 mg (e.g., about 100 mg, about 150 mg, about 200 mg, or any range between the recited values); and (c) SNAC in an amount of about 200 mg to about 400 mg (e.g., about 200 mg, about 300 mg, about 400 mg, or any range between the recited values).

In some embodiments, the small molecule drug and the large molecule drug can be present in two or more dosage forms, for example, one dosage form can contain the small molecule drug and a different dosage form can contain the large molecule drug. In some embodiments, the two or more dosage forms are all oral dosage forms.

In any of the embodiments described herein, unless otherwise specified or contrary from context, the pharmaceutical composition can be in the form of a solid oral dosage form. In some embodiments, the pharmaceutical composition herein can be presented in discrete units (which is referred to herein as “unit dosage forms” or “dosage units”), such as capsules, pills, cachets, lozenges, or tablets, each containing a predetermined amount of the active compound(s). In any of the embodiments described herein, unless otherwise specified or contrary from context, the pharmaceutical composition can be in a unit dosage form. To be clear, the pharmaceutical composition herein can include one or more dosage units. For example, the pharmaceutical composition herein can typically be a capsule or tablet, wherein each capsule or tablet constitutes a dosage unit. As used herein, each “unit dose” of the pharmaceutical composition may contain one or more unit dosage forms or dosage units, when more than one dosage units are used to satisfy the unit dose, the dosage units can be the same or different. In preferred embodiments, each unit dose contains a single dosage unit.

The pharmaceutical composition herein can optionally include one or more further excipients, such as those suitable for oral administration. For example, in some embodiments, the pharmaceutical composition herein includes at least one pharmaceutically acceptable excipient. The term “excipient” as used herein broadly refers to any component other than the active therapeutic ingredient(s). The excipient may be an inert substance, an inactive substance, and/or a not medicinally active substance. The excipient may serve various purposes, e.g. as a carrier, vehicle, filler, binder, lubricant, glidant, disintegrant, flow control agents, crystallization retarders, solubilizers, stabilizer, colouring agent, flavouring agent, surfactant, enzyme inhibitors, basifiers, acidifiers, emulsifier and/or to improve administration, and/or absorption of the active substance, tablet coating agents to control the dissolution rates of the solid dosage form according to the pH in the GI tract. A person skilled in the art may select one or more of the aforementioned excipients with respect to the particular desired properties of the solid oral dosage form by routine experimentation and without any undue burden. The amount of each excipient used may vary within ranges conventional in the art. Techniques and excipients which may be used to formulate oral dosage forms are described in Handbook of Pharmaceutical Excipients, 6th edition, Rowe et al., Eds., American Pharmaceuticals Association and the Pharmaceutical Press, publications department of the Royal Pharmaceutical Society of Great Britain (2009); and Remington: the Science and Practice of Pharmacy, 21th edition, Gennaro, Ed., Lippincott Williams & Wilkins (2005). In some embodiments the excipients may be selected from binders, such as polyvinyl pyrrolidone (povidone), etc.; fillers such as cellulose powder, microcrystalline cellulose, cellulose derivatives like hydroxymethylcellulose, hydroxyethylcellulose, hydroxypropylcellulose and hydroxy-propylmethylcellulose, dibasic calcium phosphate, corn starch, pregelatinized starch, etc.; lubricants and/or glidants such as stearic acid, magnesium stearate, sodium stearylfumarate, glycerol tribehenate, etc.; flow control agents such as colloidal silica, talc, etc.; crystallization retarders such as Povidone, etc.; solubilizers such as Pluronic, Povidone, etc.; colouring agents, including dyes and pigments such as Iron Oxide Red or Yellow, titanium dioxide, talc, etc.; pH control agents such as citric acid, tartaric acid, fumaric acid, sodium citrate, dibasic calcium phosphate, dibasic sodium phosphate, etc.; surfactants and emulsifiers such as Pluronic, polyethylene glycols, sodium carboxymethyl cellulose, polyethoxylated and hydrogenated castor oil, etc.; and mixtures of two or more of these excipients and/or adjuvants.

In some embodiments, the pharmaceutical composition herein can comprise a lubricant, a binder, a filler, and/or a chelating agent (e.g., ethylene diamine tetraacetate (EDTA)). However, in some embodiments, the pharmaceutical composition herein can also be free or substantially free of a lubricant, such as having less than 0.1% by weight, less than 0.05% by weight, less than 0.01% by weight, or non-detectable amount, of a lubricant. In some embodiments, the pharmaceutical composition herein can also be free or substantially free of a binder, such as having less than 0.1% by weight, less than 0.05% by weight, less than 0.01% by weight, or non-detectable amount, of a binder. In some embodiments, the pharmaceutical composition herein can also be free or substantially free of a filler, such as having less than 0.1% by weight, less than 0.05% by weight, less than 0.01% by weight, or non-detectable amount, of a filler. In some embodiments, the pharmaceutical composition herein can also be free or substantially free of a chelating agent, such as having less than 0.1% by weight, less than 0.05% by weight, less than 0.01% by weight, or non-detectable amount, of a chelating agent.

Method of Preparation

The pharmaceutical compositions can be prepared by those skilled in the art in view of the present disclosure.

In some embodiments, the present disclosure also provides a method of preparing a pharmaceutical composition comprising a small molecule drug (e.g., any of those described herein), which comprises:
(a) mixing the small molecule drug (e.g., any of those described herein, such as a SGLT-2 inhibitor described herein) with a compound of Formula II described herein or pharmaceutically acceptable salt thereof, and an aliphatic acid of Formula I described herein or pharmaceutically acceptable salt thereof to form a mixture; (b) freeze-drying the mixture formed in (a) to form a freeze-dried mixture; and optionally (c) mixing the freeze-dried mixture with a pharmaceutically acceptable excipient.

In some embodiments, the present disclosure also provides a method of preparing a pharmaceutical composition comprising a small molecule drug (e.g., any of those described herein), which comprises:
(a) mixing the small molecule drug (e.g., any of those described herein, such as a SGLT-2 inhibitor described herein) with a compound of Formula II described herein or pharmaceutically acceptable salt thereof, and an aliphatic acid of Formula I described herein or pharmaceutically acceptable salt thereof to form a mixture; and optionally (b) mixing the mixture with a pharmaceutically acceptable excipient.

In some embodiments, the present disclosure also provides a method of preparing a pharmaceutical composition comprising a small molecule and large molecule drug, which comprises:
(a) mixing the large molecule drug (e.g., any of those described herein, such as an incretin described herein), optionally the small molecule drug, with a compound of Formula II described herein or pharmaceutically acceptable salt thereof, and an aliphatic acid of Formula I described herein or pharmaceutically acceptable salt thereof to form a mixture; (b) freeze-drying the mixture formed in (a) to form a freeze-dried mixture; and optionally (c) mixing the freeze-dried mixture with a pharmaceutically acceptable excipient.

In some embodiments, the small molecule drug and/or the large molecule drug, as applicable, is mixed first with the compound of Formula II described herein or pharmaceutically acceptable salt thereof, followed by addition of the aliphatic acid of Formula I described herein or pharmaceutically acceptable salt thereof to form the mixture.

In some embodiments, the present disclosure also provide a method of preparing a composition comprising an incretin (e.g., any of those described herein, such as semaglutide) and a small molecule (e.g., any of those described herein, such as empagliflozin), the method comprising:
(a) mixing the incretin with a compound of Formula II:

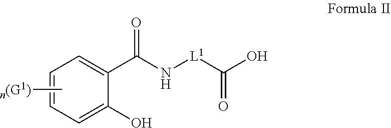

or a pharmaceutically acceptable salt thereof, wherein:
n is an integer selected from 0, 1, 2, 3, or 4;
G¹ at each occurrence is independently OH, NH₂, NH(C₁₋₄ alkyl), N(C₁₋₄ alkyl)(C₁₋₄ alkyl), halogen (e.g., Cl), C₁₋₄ alkyl, or C₁₋₄ alkoxy (e.g., OCH₃); and
L¹ is a substituted or unsubstituted C₂-C₁₆ alkylene, or substituted or unsubstituted C₂-C₁₆ alkenylene; and
(b) freeze-drying the mixture formed in (a).

In some embodiments, the mixing in (a) further comprises mixing the incretin, optionally the small molecule, and the compound of Formula II or pharmaceutically acceptable salt thereof, and an aliphatic acid of Formula I: RCOOH, wherein R represents an aliphatic group, or a pharmaceutically acceptable salt thereof. In some embodiments, the aliphatic acid of Formula I is a linear aliphatic acid having 2 to 20 carbon atoms, such as caprylic acid, capric acid, or lauric acid. In some embodiments, the weight ratio of (i) the aliphatic acid of Formula I or pharmaceutically acceptable salt thereof to (ii) the compound of Formula II or pharmaceutically acceptable salt thereof, (i)/(ii), ranges from about 20:1 to about 1:20, such as 5:1 to 1:5, e.g., about 1:2. In some embodiments, the aliphatic acid of Formula I or pharmaceutically acceptable salt thereof is in an amount of about 50 mg to about 300 mg. In some embodiments, the compound of Formula II or pharmaceutically acceptable salt thereof is in an amount of about 200 mg to about 400 mg.

In some specific embodiments, in the methods above as applicable, (a) the small molecule drug (e.g., any of those described herein) is in an amount of about 1 mg to about 1000 mg (e.g., about 10 mg, about 50 mg, about 100 mg, or any range between the recited values); (b) the aliphatic acid of Formula I or pharmaceutically acceptable salt thereof is sodium caprate, in an amount of about 50 mg to about 300 mg (e.g., about 100 mg, about 150 mg, about 200 mg, or any range between the recited values); and/or (c) the compound of Formula II or pharmaceutically acceptable salt thereof is SNAC, in an amount of about 200 mg to about 400 mg (e.g., about 200 mg, about 300 mg, about 400 mg, or any range between the recited values).

In some specific embodiments, in the methods above as applicable, (a) the large molecule drug (e.g., any of those described herein) is an incretin, for example, in an amount of about 1 mg to about 200 mg (e.g., about 10 mg, about 50 mg, about 100 mg, or any range between the recited values), and the small molecule is in an amount of about 1 mg to about 1000 mg (e.g., about 1 mg, 10 mg, about 50 mg, about 100 mg, or any range between the recited values); (b) the aliphatic acid of Formula I or pharmaceutically acceptable salt thereof is sodium caprate, in an amount of about 50 mg to about 300 mg (e.g., about 100 mg, about 150 mg, about 200 mg, or any range between the recited values); and/or (c) the compound of Formula II or pharmaceutically acceptable salt thereof is SNAC, in an amount of about 200 mg to about 400 mg (e.g., about 200 mg, about 300 mg, about 400 mg, or any range between the recited values).

In some specific embodiments, in the methods above as applicable, (a) the large molecule drug is a GLP-1 receptor agonist (e.g., any of those described herein), for example, in an amount of about 1 mg to about 200 mg (e.g., about 10 mg, about 50 mg, about 100 mg, or any range between the recited values), and the small molecule drug is an SGLT2 inhibitor (e.g., any of those described herein), for example, in an amount of about 1 mg to about 200 mg (e.g., about 10 mg, about 50 mg, about 100 mg, or any range between the recited values); (b) the aliphatic acid of Formula I or pharmaceutically acceptable salt thereof is sodium caprate, in an amount of about 50 mg to about 300 mg (e.g., about 100 mg, about 150 mg, about 200 mg, or any range between the recited values); and/or (c) the compound of Formula II or pharmaceutically acceptable salt thereof is SNAC, in an amount of about 200 mg to about 400 mg (e.g., about 200 mg, about 300 mg, about 400 mg, or any range between the recited values).

In some specific embodiments, in the methods above as applicable, (a) the large molecule drug is semaglutide in an amount of about 1 mg to about 200 mg (e.g., about 3 mg, about 10 mg, about 50 mg, about 100 mg, or any range between the recited values), and the small molecule drug is empagliflozin in an amount of about 1 mg to about 200 mg (e.g., about 5 mg, 10 mg, about 20 mg, about 100 mg, or any range between the recited values); (b) the aliphatic acid of Formula I or pharmaceutically acceptable salt thereof is sodium caprate, in an amount of about 50 mg to about 300 mg (e.g., about 100 mg, about 150 mg, about 200 mg, or any range between the recited values); and/or (c) the compound of Formula II or pharmaceutically acceptable salt thereof is SNAC, in an amount of about 200 mg to about 400 mg (e.g., about 200 mg, about 300 mg, about 400 mg, or any range between the recited values).

The composition comprising the therapeutic agent prepared by the method herein is also a novel composition of the present disclosure.

In some embodiments, the present disclosure further provides a method of preparing a pharmaceutical composition comprising mixing the composition comprising the therapeutic agent prepared by the method herein with a pharmaceutically acceptable excipient (e.g., any of those described herein).

### Method of Treatment

The pharmaceutical compositions herein can be used for treating or preventing any diseases or disorders for which administering the small molecule drug and/or the large molecule drug is beneficial, which include any of those known indications that can be treated or prevented by administering the small molecule drug and/or the large molecule drug and any of those indications for which the small molecule drug and/or the large molecule drug have been approved for marketing by a regulatory agency, such as the U.S. Food and Drug Administration and the alike. For example, in some embodiments, the pharmaceutical composition comprises empagliflozin, and the pharmaceutical composition can be used for treating or preventing any diseases or disorders known to be beneficial by administering empagliflozin, such as type 2 diabetes, obesity, liver disease (such as NASH), renal disease, etc., which include any of those approved indications for empagliflozin by the U.S. Food and Drug Administration and the alike, and also include those known such as those described in U.S. Pat. Nos. 8,551,957, 9,949,997, 9,949,998, 10,258,637, and 11,090,323. In some embodiments, the pharmaceutical composition comprises a GLP-1 RA, such as semaglutide, and the pharmaceutical composition can be used for treating or preventing any diseases or disorders known to be beneficial by administering a GLP-1 RA such as semaglutide, for example, type 2 diabetes, obesity, NASH (non-alcoholic steatohepatitis), etc., which include any of those approved indications for GLP-1 RAs, such as for semaglutide, by the U.S. Food and Drug Administration and the alike, and also include those known.

### Combination Therapies

In some embodiments, the pharmaceutical compositions herein can be used as a combination therapy. For example, in some embodiments, the pharmaceutical composition can be a fixed-dose combination of two or more active therapeutic agents. Non-limiting combination therapies contemplated include the combination of GLP-1 RA and SGLT2 inhibitors.

In some embodiments, the present disclosure provides a method of treating type 2 diabetes in a subject in need thereof, the method comprising administering to the subject the pharmaceutical composition herein, which comprises a fixed-dose combination comprising an SGLT2 inhibitor and an incretin. In some preferred embodiments, the method comprises administering to the subject the pharmaceutical composition herein, which comprises a fixed-dose combination comprising an SGLT2 inhibitor and a GLP-1 RA, preferably, a fixed-dose combination comprising a therapeutically effective amount of empagliflozin and semaglutide.

In some embodiments, the present disclosure provides a method of treating obesity in a subject in need thereof, the method comprising administering to the subject the pharmaceutical composition herein, which comprises a fixed-dose combination comprising an SGLT2 inhibitor and an incretin. In some preferred embodiments, the method comprises administering to the subject the pharmaceutical composition herein, which comprises a fixed-dose combination comprising an SGLT2 inhibitor and a GLP-1 RA, preferably, a fixed-dose combination comprising a therapeutically effective amount of empagliflozin and semaglutide.

In some embodiments, the present disclosure provides a method of treating a renal disease such as chronic kidney disease (CKD) in a subject in need thereof, the method comprising administering to the subject the pharmaceutical composition herein, which comprises a fixed-dose combination comprising an SGLT2 inhibitor and an incretin. In some preferred embodiments, the method comprises administering to the subject the pharmaceutical composition herein, which comprises a fixed-dose combination comprising an SGLT2 inhibitor and a GLP-1 RA, preferably, a fixed-dose combination comprising a therapeutically effective amount of empagliflozin and semaglutide.

In some embodiments, the present disclosure provides a method of treating a liver disease such as NASH in a subject in need thereof, the method comprising administering to the subject the pharmaceutical composition herein, which comprises a fixed-dose combination comprising an SGLT2 inhibitor and an incretin. In some preferred embodiments, the method comprises administering to the subject the pharmaceutical composition herein, which comprises a fixed-dose combination comprising an SGLT2 inhibitor and a GLP-1 RA, preferably, a fixed-dose combination comprising a therapeutically effective amount of empagliflozin and semaglutide.

In some embodiments, the present disclosure provides a method of reducing the risk of cardiovascular death in a subject with type 2 diabetes mellitus, the method comprising administering to the subject the pharmaceutical composition herein, which comprises a fixed-dose combination comprising an SGLT2 inhibitor and an incretin. In some preferred embodiments, the method comprises administering to the subject the pharmaceutical composition herein, which comprises a fixed-dose combination comprising an SGLT2 inhibitor and a GLP-1 RA, preferably, a fixed-dose combination comprising a therapeutically effective amount of empagliflozin and semaglutide.

In some embodiments, the present disclosure provides a method of reducing the risk of hospitalization for heart failure in a subject with type 2 diabetes mellitus, the method comprising administering to the subject the pharmaceutical composition herein, which comprises a fixed-dose combination comprising an SGLT2 inhibitor and an incretin. In some preferred embodiments, the method comprises administering to the subject the pharmaceutical composition herein, which comprises a fixed-dose combination comprising an SGLT2 inhibitor and a GLP-1 RA, preferably, a fixed-dose combination comprising a therapeutically effective amount of empagliflozin and semaglutide.

In some embodiments, the present disclosure provides a method of reducing the risk of all-cause mortality in a subject with type 2 diabetes mellitus, the method comprising administering to the subject the pharmaceutical composition herein, which comprises a fixed-dose combination comprising an SGLT2 inhibitor and an incretin. In some preferred embodiments, the method comprises administering to the subject the pharmaceutical composition herein, which comprises a fixed-dose combination comprising an SGLT2 inhibitor and a GLP-1 RA, preferably, a fixed-dose combination comprising a therapeutically effective amount of empagliflozin and semaglutide.

In some embodiments, the present disclosure provides a method of treating type 2 or type 1 diabetes mellitus in a subject with or at risk of oxidative stress, vascular stress and/or endothelial dysfunction, or diseases or conditions related or associated therewith, the method comprising administering to the subject the pharmaceutical composition herein, which comprises a fixed-dose combination comprising an SGLT2 inhibitor and an incretin. In some preferred embodiments, the method comprises administering to the subject the pharmaceutical composition herein, which comprises a fixed-dose combination comprising an SGLT2 inhibitor and a GLP-1 RA, preferably, a fixed-dose combination comprising a therapeutically effective amount of empagliflozin and semaglutide.

In some embodiments, the present disclosure provides a method of preventing, reducing the risk of or delaying the occurrence of a cardiovascular event in a subject with type 2 or type 1 diabetes mellitus or with pre-diabetes, the method comprising administering to the subject the pharmaceutical composition herein, which comprises a fixed-dose combination comprising an SGLT2 inhibitor and an incretin. In some preferred embodiments, the method comprises administering to the subject the pharmaceutical composition herein, which comprises a fixed-dose combination comprising an SGLT2 inhibitor and a GLP-1 RA, preferably, a fixed-dose combination comprising a therapeutically effective amount of empagliflozin and semaglutide.

In some embodiments, the present disclosure provides a method of reducing arterial stiffness in a subject, the method comprising administering to the subject the pharmaceutical composition herein, which comprises a fixed-dose combination comprising an SGLT2 inhibitor and an incretin. In some preferred embodiments, the method comprises administering to the subject the pharmaceutical composition herein, which comprises a fixed-dose combination comprising an SGLT2 inhibitor and a GLP-1 RA, preferably, a fixed-dose combination comprising a therapeutically effective amount of empagliflozin and semaglutide.

In some embodiments, the present disclosure provides a method of improving glycemic control in a subject with type 2 diabetes mellitus, the method comprising administering to the subject the pharmaceutical composition herein, which comprises a fixed-dose combination comprising an SGLT2 inhibitor and an incretin. In some preferred embodiments, the method comprises administering to the subject the pharmaceutical composition herein, which comprises a fixed-dose combination comprising an SGLT2 inhibitor and a GLP-1 RA, preferably, a fixed-dose combination comprising a therapeutically effective amount of empagliflozin and semaglutide.

The administering of the pharmaceutical composition herein for the methods herein is typically an oral administration. The daily doses of the SGLT2 inhibitor and/or the GLP-1 RA, such as that of empagliflozin and semaglutide, can be determined by methods known in the art. For example, in some embodiments, the daily dose of the SGLT2 inhibitor, such as empagliflozin, can range from 1 mg to 100 mg, such as about 5 mg, about 10 mg, about 25 mg, about 50 mg, or any range in between. In some embodiments, the daily dose of the GLP-1 RA, such as semaglutide, can range from about 1 mg to about 200 mg (e.g., about 3 mg, about 10 mg, about 50 mg, about 100 mg, or any range between the recited values).

GLP-1 RA and Sodium-glucose cotransporter 2 (SGLT2) proteins function independently of insulin in regulation of glucose. Sodium-glucose cotransporter 1 (SGLT1) proteins are high affinity and low-capacity transporters of glucose and are expressed in the small intestines as well as the proximal tubule of the kidneys. The SGLT1 proteins in the proximal convoluted tubule of the kidneys are responsible for less than 10% of filtered glucose reabsorption. Sodium-glucose cotransporter-2 (SGLT2) proteins are expressed in the proximal convoluted tubule of the kidneys and are responsible for roughly 90% of filtered glucose reabsorption (Scheen AJ. Pharmacodynamics, efficacy and safety of sodium-glucose co-transporter type 2 (SGLT2) inhibitors for the treatment of type 2 diabetes mellitus. Drugs. 2015 Jan.; 75(1):33-59.). There are several SGLT2 selective inhibitors approved by FDA. Including canagliflozin, dapagliflozin, and empagliflozin. Of the three FDA approved drugs, empagliflozin has the greatest selectivity for SGLT2 compared to SGLT1, while canagliflozin is the least selective (Shubrook J H, Bokaie B B, Adkins S E. Empagliflozin in the treatment of type 2 diabetes: evidence to date. Drug design, development and therapy. 2015; 9:5793-803.). SGLT2 inhibitors have demonstrated clinically body weight control and antihypertensive benefits. The risk of hypoglycemia with SGLT2 inhibitors is small when compared to insulin and sulfonylureas. (Desouza C V, Gupta N, Patel A. Cardiometabolic Effects of a New Class of Antidiabetic Agents. Clin Ther. 2015 Jun. 1; 37(6):1178-94.)

Recent studies demonstrated that there was no increased risk for major adverse cardiovascular events with GLP-1 RAs, such as dapagliflozin and empagliflozin. More importantly, studies showed that in T2D patients with high risk of cardiovascular disease events, SGLT2 inhibition demonstrated significant CV benefits, such as a 38% relative risk reduction in death from cardiovascular causes in the empagliflozin group versus the placebo group (Zinman B, Wanner C, Lachin J M, Fitchett D, Bluhmki E, Hantel S, et al. Empagliflozin, Cardiovascular Outcomes, and Mortality in Type 2 Diabetes. The New England journal of medicine. 2015 Nov. 26; 373(22):2117-28. This was a pivotal cardiovascular outcomes trial for empagliflozin.).

GLP-1RAs and SGLT-2 inhibitors showed the evidence to improve clinical outcomes in diabetic patients with cardiovascular diseases. The new T2D pharmacotherapy guidelines have recommended the use of GLP-1RAs for prevention and treatment of obese patients with risks of atherosclerotic cardiovascular diseases, whereas SGLT-2 is has been proposed for patients with a risk of chronic heart failure. However, there are no systemic research, especially clinical trials to further evaluate the potential additive or synergistic effects of these two classes of medications for the CV benefits, given both classes of drugs have different modes of mechanisms for treatments of T2D and diabetes.

The key reason of not having robust clinical research on the combinations of GLP-1 RA and SGLT2i may be explained on the commercial or financial basis. GLP-1 RA are also injectable peptides daily or weekly (except for oral semaglutide) while SGLT2i are all oral tablets administered daily. It is not feasible to develop a financially supportive combination, since such injectable and oral combinations may not be acceptable to patients in practice, and more importantly, no intellectual properties can be obtained for such direct combinations of two commercially available medications.

Commercially, the only available oral GLP-1 RA is Rybelsus, which however, has too low BA %, which makes it economically challenging in combinations. Technically, oral semaglutide is absorbed in stomach facilitated by the carrier, SNAC, while SGLT2 inhibitor are formulated in tablets with coated film, which target absorption in small intestine, where the absorption area is large and the villi of endothelial cells are abundant, allowing much higher permeability than in stomach.

In some embodiments, the present invention enables the co-formulations of GLP-1 RA and SGLT2 inhibitor into the same tablets, which showed that these two different types of molecules with distinct physic-chemical, biological, metabolic properties not only do not interfere with absorptions of each entity, but also enhance the absorption and improve the time-course pharmacokinetic profiles of small molecules, SGLT2 inhibitors here. Such fixed-dose combination of GLP-1 RA and SGLT2 inhibitors in one tablet brings significant medical values with convenient use and economic feasibility.

### Definitions

As used herein, the singular form “a”, “an”, and “the”, includes plural references unless it is expressly stated or is unambiguously clear from the context that such is not intended.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Headings and subheadings are used for convenience and/or formal compliance only, do not limit the subject technology, and are not referred to in connection with the interpretation of the description of the subject technology. Features described under one heading or one subheading of the subject disclosure may be combined, in various embodiments, with features described under other headings or subheadings. Further it is not necessarily the case that all features under a single heading or a single subheading are used together in embodiments.

As used herein, the term “about” modifying an amount related to the invention refers to variation in the numerical quantity that can occur, for example, through routine testing and handling; through inadvertent error in such testing and handling; through differences in the manufacture, source, or purity of ingredients employed in the invention; and the like. As used herein, “about” a specific value also includes the specific value, for example, about 10% includes 10%. Whether or not modified by the term “about”, the claims include equivalents of the recited quantities. In one embodiment, the term “about” means within 20% of the reported numerical value.

The term “polypeptide” and “peptide” as used herein means a compound composed of at least five constituent amino acids connected by peptide bonds. The constituent amino acids may be from the group of the amino acids encoded by the genetic code and they may be natural amino acids which are not encoded by the genetic code, as well as synthetic amino acids.

The term “analogue” as used herein referring to a polypeptide means a modified peptide wherein one or more amino acid residues of the peptide have been substituted by other amino acid residues and/or wherein one or more amino acid residues have been deleted from the peptide and/or wherein one or more amino acid residues have been deleted from the peptide and or wherein one or more amino acid residues have been added to the peptide.

The term “derivative” as used herein in relation to a peptide means a chemically modified peptide or an analogue thereof, wherein at least one substituent is not present in the unmodified peptide or an analogue thereof, i.e. a peptide which has been covalently modified. Typical modifications are amides, 20 carbohydrates, alkyl groups, acyl groups, esters and the like. An example of a derivative of GLP-1(7-37) is NE26-((4S)-4-(hexadecanoylamino)-carboxy-butanoyl)[Arg34, Lys26] GLP-1-(7-37).

In some embodiments the term “GLP-1 analogue” as used herein refers to a peptide, or a compound, which is a variant of the human Glucagon-Like Peptide-1 (GLP-1(7-37)). GLP-1(7-37) has the sequence HAEGTFTSDV SSYLEGQAAKEFIAWLVKGRG (SEQ ID No: 1). In some embodiments the term “variant” refers to a compound which comprises one or more amino acid substitutions, deletions, additions and/or insertions.

In some embodiments, the GLP-1 receptor agonist exhibits at least 60%, 65%, 70%, 80% or 90% sequence identity to GLP-1(7-37) over the entire length of GLP-1(7-37). As an example of a method for determination of sequence identity between two analogues the two peptides [Aib8]GLP-1(7-37) and GLP-1(7-37) are aligned. The sequence identity of [Aib8]GLP-1(7-37) relative to GLP-1(7-37) is given by the number of aligned identical residues minus the number of different residues divided by the total number of residues in GLP-1(7-37). Accordingly, in said example the sequence identity is (31-1)/31.

The term “GLP-1 receptor agonist” as used herein refers to a compound, which fully or partially activates the human GLP-1 receptor. In some embodiments, the GLP-1 receptor agonist is a GLP-1 analogue, optionally comprising one substituent. In some embodiments the GLP-1 receptor agonist is exendin-4, the sequence of which is HGEGTFITSDL SKQMEEEAVR-LFIEWLKNGGPSSGAPPPS (SEQ ID No: 2). In some embodiments the GLP-1 receptor agonist comprises one substituent which is covalently attached to the peptide. In some embodiments the substituent comprises a fatty acid or a fatty diacid. In some embodiments the substituent comprises a C16, C18 or C20 fatty acid. In some embodiments the substituent comprises a C16, C18 or C20 fatty diacid. Examples of GLP-1 RA include but not limited to semaglutide, liraglutide, dulaglutide, lixisenatide, exenatide and others.

In some embodiments, the GLP-1 receptor agonist is selected from one or more of the GLP-1 receptor agonists disclosed in WO93/19175, WO96/29342, WO98/08871, WO99/43707, WO99/43706, WO99/43341, WO99/43708, WO2005/027978, WO2005/058954, WO2005/058958, WO2006/005667, WO2006/037810, WO2006/037811, WO2006/097537, WO2006/097538, WO2008/023050, WO2009/030738, WO2009/030771 and WO2009/030774.

In some embodiments, the GLP-1 receptor agonist is selected from the group consisting of N-epsilon37{2-[2-(2-{2-[2-((R)-3-carboxy-3-{[1-(19-carboxynonadecanoyl) piperidine-4-carbonyl]amino}propionylamino)ethoxy]ethoxy}acetylamino)ethoxy]ethoxy}acetyl [desaminoHis7,Glu22,Arg26,Arg34,Lys37]GLP-1(7-37)amide; N-epsilon26{2-[2-(2-{2-[2-((R)-3-carboxy-3-{[1-(19-carboxynonadecanoyl) piperidine-4-carbonyl]amino}propionylamino)ethoxy]ethoxy}acetylamino)ethoxy] ethoxy}acetyl [desaminoHis7, Arg34] GLP-1-(7-37); N-epsilon37{2-[2-(2-{2-[2-((S)-3-carboxy-3-{[1-(19-carboxynonadecanoyl) piperidine-4-carbonyl]amino}propionylamino)ethoxy]ethoxy}acetylamino)ethoxy] ethoxy}acetyl[Aib8,Glu22,Arg26,Arg34,Lys37]GLP-1-(7-37)amide; N-epsilon37-[2-(2-[2-(2-[2-(2-((R)-3-[1-(17-carboxyheptadecanoyl)piperidin-4-ylcarbonylamino]3-carboxypropionylamino)ethoxy)ethoxy]acetylamino)ethoxy]ethoxy)acetyl] [,DesaminoHis7, Glu22 Arg26, Arg34, Phe(m-CF3)28]GLP-1-(7-37)amide; N-epsilon26-[(S)-4-carboxy-4-({trans-4-[(19-carboxynonadecanoylamino)methyl] cyclohexanecarbonyl}amino)butyryl][Aib8,Arg34]GLP-1-(7-37); N-epsilon26-{4-[(S)-4-carboxy-4-({trans-4-[(19-carboxynonadecanoylamino) methyl]cyclohexanecarbonyl}amino)butyrylamino]butyryl}[Aib8,Arg34]GLP-1-(7-37); N-epsilon26-[2-(2-{2-[(S)-4-carboxy-4-({trans-4-[(19-carboxy-nonadecanoylamino) methyl]cyclohexanecarbonyl}amino)butyrylamino]ethoxy}ethoxy)acetyl][Aib8,Arg34]GLP-1-(7-37); N-epsilon26-[2-(2-{2-[2-(2-{2-[(S)-4-carboxy-4-({trans-4-[(19-carboxy-nonadecanoylamino)methyl]cyclohexanecarbonyl}amino)butyrylamino]ethoxy}ethoxy)acety lamino]ethoxy}ethoxy) acetyl] [Aib8,Arg34]GLP-1-(7-37)amide; N-epsilon37-[2-(2-{2-[2-(2-{2-[(S)-4-carboxy-4-({trans-4-[(19-carboxy-nonadecanoylamino)methyl]cyclohexanecarbonyl}amino) butyrylamino]ethoxy}ethoxy)acetylamino]ethoxy}ethoxy)acetyl] [Aib8,Glu22,Arg26, Arg34,Lys37]GLP-1-(7-37)amide; N-epsilon37-[2-(2-{2-[2-(2-{2-[(S)-4-carboxy-4-({trans-4-[(19-carboxy-nonadecanoylamino)methyl]cyclohexanecarbonyl}amino) butyrylamino]ethoxy}ethoxy)acetylamino]ethoxy}ethoxy)acetyl] [DesaminoHis7,Glu22, Arg26,Arg34,Lys37]GLP-1-(7-37)amide; N-epsilon37-[2-(2-{2-[2-(2-{2-[(S)-4-carboxy-4-({4-[(trans-19-carboxy-nonadecanoylamino)methyl]cyclohexanecarbonyl}amino) butyrylamino]ethoxy}ethoxy)acetylamino]ethoxy}ethoxy)acetyl][DesaminoHis7,Arg26,Arg34,Lys 37]GLP-1-(7-37)amide; N-epsilon37-[2-(2-{2-[2-(2-{2-[(S)-4-carboxy-4-({trans-4-[(19-carboxy-nonadecanoylamino)methyl]cyclohexanecarbonyl}amino) butyrylamino]ethoxy}ethoxy)acetylamino]ethoxy}ethoxy)acetyl][DesaminoHis7,Glu22,Arg26,Arg 34,Lys37]GLP-1-(7-37); N-epsilon26[2-(2-{2-[2-(2-{2-[(S)-4-carboxy-4-({4-[(19-carboxy-nonadecanoylamino)methyl]cyclohexanecarbonyl}amino)butyrylamino] ethoxy}ethoxy) acetylamino]ethoxy}ethoxy)acetyl[Aib8, Lys 26]GLP-1 (7-37)amide; N-epsilon26 [2-(2-[2-(2-[2-(2-((S)-2-[trans-4-((9-carboxynonadecanoylamino] methyl) cyclohexylcarbonylamino]-4-carboxybutanoylamino)ethoxy)ethoxy]acetylamino) ethoxy]ethoxy)acetyl] [Aib8, Lys26]GLP-1 (7-37)amide; N-epsilon37-[2-(2-{2-[2-(2-{2-[(S)-4-carboxy-4-({trans-4-[(19-carboxy-nonadecanoylamino)methyl]cyclohexanecarbonyl}amino)butyrylamino]ethoxy}ethoxy)acetylamino]ethoxy}ethoxy)acetyl][DesaminoHis7,Arg 26,Arg34,Lys37]GLP-1-(7-37); N-epsilon37-[2-(2-{2-[2-(2-{2-[(S)-4-carboxy-4-({trans-4-[(19-carboxy-nonadecanoylamino)methyl]cyclohexanecarbonyl}amino)butyrylamino]ethoxy}ethoxy)acety lamino]ethoxy}ethoxy)acetyl] [DesaminoHis7,Glu22,Arg26,Glu30,Arg34,Lys37]GLP-1-(7-37); N-epsilon26-[2-(2-{2-[(S)-4-carboxy-4-((S)-4-carboxy-4-{4-[4-(16-(1H-tetrazol-5-yl)-hexadecanoylsulfamoyl)butyrylamino]-butyrylamino}butyrylamino) butyrylamino]ethoxy}ethoxy)acetyl] [Aib8,Arg34]GLP-1-(7-37); N-epsilon26-[2-(2-{2-[(S)-4-carboxy-4-((S)-4-carboxy-4-{12-[4-(16-(1H-tetrazol-5-yl)hexadecanoyl-sulfamoyl)butyrylamino]dodecanoylamino}butyrylamino) butyrylamino]ethoxy}ethoxy)acetyl] [Aib8,Arg34]GLP-1-(7-37); N-epsilon26-[2-(2-{2-[(S)-4-carboxy-4-((S)-4-carboxy-4-{6-[4-(16-(1H-tetrazol-5-yl)hexadecanoyl-sulfamoyl)butyrylamino]hexanoylamino} butyrylamino)butyrylamino]ethoxy}ethoxy) acetyl] [Aib8,Arg34]GLP-1-(7-37); N-epsilon26-[2-(2-{2-[(S)-4-carboxy-4-((S)-4-carboxy-4-{4-[4-(16-(1H-tetrazol-5-yl)hexadecanoylsulfamoyl)butyrylamino]butyrylamino}butyrylamino)butyrylamino]ethoxy}ethoxy)acetyl] [Aib8,Arg34]GLP-1-(7-34); N-epsilon26-[2-(2-{2-[(S)-4-carboxy-4-((S)-4-carboxy-4-{12-[4-(16-(1H-tetrazol-5-yl)hexadecanoylsulfamoyl)butyrylamino]-dodecanoylamino}butyrylamino) butyrylamino] ethoxy}ethoxy)acetyl] [Aib8,Arg34]GLP-1-(7-34); N-epsilon26-[2-(2-{2-[(S)-4-carboxy-4-((S)-4-carboxy-4-{6-[4-(16-(1H-tetrazol-5-yl)hexadecanoylsulfamoyl) butyrylamino]hexanoylamino}butyrylamino) butyrylamino]ethoxy}ethoxy)acetyl][Aib8,Arg34]GLP-1-(7-34); N-epsilon26-[2-(2-{2-[(S)-4-carboxy-4-((S)-4-carboxy-4-{12-[4-(16-(1H-tetrazol-5-yl)hexadecanoyl-sulfamoyl)butyrylamino]dodecanoylamino}butyrylamino)butyrylamino]ethoxy}ethoxy)acety 1][Aib8,Arg34]GLP-1-(7-35); N-epsilon26-[2-(2-{2-[(S)-4-carboxy-4-((S)-4-carboxy-4-{6-[4-(16-(1H-tetrazol-5-yl)hexadecanoylsulfamoyl)butyrylamino]hexanoylamino}butyrylamino)butyrylamino]ethoxy}ethoxy)acetyl][Aib8,Arg34]GLP-1-(7-35); N-epsilon26-[2-(2-{2-[(S)-4-carboxy-4-((S)-4-carboxy-4-{6-[4-(16-(1H-tetrazol-5-yl)hexadecanoylsulfamoyl)butyrylamino]hexanoylamino}butyrylamino)butyrylamino]ethoxy}ethoxy)acetyl][Aib8,Arg34]GLP-1-(7-36)amide; N-epsilon26-[2-(2-{2-[(S)-4-carboxy-4-((S)-4-carboxy-4-{6-[4-(16-(1H-tetrazol-5-yl)hexadecanoylsulfamoyl)butyrylamino]hexanoylamino}butyrylamino) butyrylamino]ethoxy}ethoxy)acetyl][Aib8,Arg34]GLP-1-(7-35); N-epsilon26-[2-(2-{2-[(S)-4-carboxy-4-((S)-4-carboxy-4-{12-[4-(16-(1H-tetrazol-5-yl)hexadecanoylsulfamoyl)butyrylamino]dodecanoylamino}butyryl-amino)butyrylamino]ethoxy}ethoxy)acetyl][Aib8,Lys33,Arg34]GLP-1-(7-34); N-epsilon26-[2-(2-{2-[(S)-4-carboxy-4-((S)-4-carboxy-4-{12-[4-(16-(1H-tetrazol-5-yl)hexadecanoylsulfamoyl)butyrylamino]dodecanoylamino}butyrylamino)butyrylamino]etho xy}ethoxy)acetyl][Aib8,Arg34]GLP-1-(7-36)amide; N-epsilon26-[2-(2-{2-[2-(2-{2-[2-(2-{2-[2-(2-{2-[2-(2-{2-[2-(2-{2-f(S)-4-carboxy-4-((S)-4-carboxy-4-{112-[4-(16-(1H-tetrazol-5-yl)hexadecanoylsulfamoyl) butyrylamino]dodecanoylamino}butyrylamino) butyrylamino]ethoxy}ethoxy) acetylamino]ethoxy}ethoxy)acetylamino]ethoxy}ethoxy)acetylamino]ethoxy}ethoxy)acetyla mino]ethoxy}ethoxy)acetylamino]ethoxy}ethoxy)acetyl] [Aib8,Lys26,Arg34]GLP-1-(7-36)amide; N-epsilon37-[2-(2-{2-[(S)-4-carboxy-4-((S)-4-carboxy-4-{12-[4-(16-(1H-tetrazol-5-yl)hexadecanoylsulfamoyl)butyrylamino] dodecanoylamino}butyrylamino) butyrylamino]ethoxy}ethoxy)acetyl][Aib8,Glu22,Arg26,Arg34,Lys37]GLP-1-(7-37)amide; N-epsilon37-[2-(2-{2-[(S)-4-carboxy-4-((S)-4-carboxy-4-{12-[4-(16-(1H-tetrazol-5-yl)hexadecanoylsulfamoyl)butyrylamino]dodecanoylamino}butyrylamino) butyrylamino]ethoxy}ethoxy)acetyl][DesaminoHis7,Glu22,Arg26,Arg34,Lys37]GLP-1-(7-37)amide; N-epsilon37{2-[2-(2-{2-[2-((R)-3-carboxy-3-{1[1-(19-carboxy-nonadecanoyl)piperidine-4-carbonyl]amino}propionylamino)ethoxy]ethoxy}acetylamino)ethoxy] ethoxy}acetyl [desaminoHis7,Glu22,Arg26,Arg34,Lys37]GLP-1(7-37)amide; N-epsilon37{2-[2-(2-{2-[2-((S)-3-carboxy-3-{[1-(19-carboxynonadecanoyl)piperidine-4-carbonyl]amino}propionylamino) ethoxy]ethoxy}acetylamino)ethoxy] ethoxy} acetyl [Aib8,Glu22, Arg26,Arg34, Lys37]GLP-1-(7-37)amide; N-epsilon37-[2-(2-[2-(2-[2-(2-((R)-3-[1-(17-carboxyhepta-decanoyl)piperidin-4-ylcarbonylamino]3-carboxy-propionylamino)ethoxy)ethoxy]acetylamino) ethoxy] ethoxy)acetyl] [DesaminoHis7, Glu22,Arg26, Arg34,Phe(m-CF3)28]GLP-1-(7-37)amide; N-epsilon37-[2-(2-{2-[2-(2-{2-[(S)-4-carboxy-4-({trans-4-[(19-carboxy-nonadecanoylamino)methyl] cyclohexanecarbonyl}amino)butyrylamino]ethoxy}ethoxy)acetylamino] ethoxy}ethoxy)acetyl][Aib8,Glu22,Arg26,Arg34,Lys37]GLP-1-(7-37)amide; N-epsilon37-[2-(2-{2-[2-(2-{2-[(S)-4-carboxy-4-({trans-4-[(19-carboxy-nonadecanoylamino)methyl]cyclohexanecarbonyl} amino)butyrylamino]ethoxy}ethoxy) acetylamino]ethoxy}ethoxy)acetyl][DesaminoHis7,Glu22,Arg26,Arg34,Lys37]GLP-1-(7-37)amide; N-epsilon37-[2-(2-{2-[2-(2-{2-[(S)-4-carboxy-4-({trans-4-[(19-carboxy-nonadecanoylamino)methyl]cyclohexanecarbonyl}amino)butyrylamino]ethoxy}ethoxy)acetylamino]ethoxy}ethoxy)acetyl][DesaminoHis7,Glu22,Arg26,Arg34, Lys37]GLP-1-(7-37); N-epsilon37-[2-(2-{2-[2-(2-{2-[(S)-4-carboxy-4-({trans-4-[(19-carboxy-nonadecanoylamino) methyl]cyclohexanecarbonyl}amino)butyrylamino]ethoxy}ethoxy)acetylamino]ethoxy}ethoxy)acetyl][DesaminoHis7,Glu22,Arg26,Glu30,Arg34, Lys37]GLP-1-(7-37); N-epsilon37-[2-(2-{2-[(S)-4-carboxy-4-((S)-4-carboxy-4-{12-[4-(16-(1H-tetrazol-5-yl)hexadecanoylsulfamoyl) butyrylamino]dodecanoylamino}butyrylamino) butyrylamino]ethoxy}ethoxy)acetyl] [Aib8,Glu22,Arg26,Arg34,Lys37]GLP-1-(7-37)amide; N-epsilon37-[2-(2-{2-[(S)-4-carboxy-4-((S)-4-carboxy-4-{12-[4-(16-(1H-tetrazol-5-yl)hexadecanoylsulfamoyl) butyrylamino]dodecanoylamino}butyrylamino) butyrylamino]ethoxy}ethoxy)acetyl] [DesaminoHis7,Glu22,Arg26,Arg34,Lys37]GLP-1-(7-37)amide; N-epsilon37-(3-((2-(2-(2-(2-(2-Hexadecyloxyethoxy)ethoxy)ethoxy) ethoxy) ethoxy)) propionyl) [DesaminoHis7,Glu22,Arg26,Arg34,Lys37]GLP-1(7-37)-amide; N-epsilon37-{2-(2-(2-(2-[2-(2-(4-(hexadecanoylamino)-4-carboxybutyryl-amino)ethoxy) ethoxy]acetyl)ethoxy)ethoxy)acetyl)}-[desaminoHis7,Glu22,Arg26, Glu30,Arg34,Lys37]GLP-1-(7-37)amide; N-epsilon37-{2-(2-(2-(2-[2-(2-(4-(hexadecanoylamino)-4-carboxybutyryl-amino) ethoxy)ethoxy]acetyl)ethoxy)ethoxy) acetyl)}-[desaminoHis7,Glu22, Arg26, Arg34,Lys37]GLP-1-(7-37)amide; N-epsilon37-(2-(2-(2-(2-(2-(2-(2-(2-(2-(octadecanoyl-amino)ethoxy)ethoxy) acetylamino)ethoxy) ethoxy)acetylamino) ethoxy)ethoxy) acetyl)[desaminoHis7,Glu22,Arg26,Arg34,Lys37] GLP-1 (7-37)amide; N-epsilon37-[4-(16-(1H-Tetrazol-5-yl)hexadecanoylsulfamoyl)butyryl][DesaminoHis7,Glu22,Arg26, Arg34, Lys37]GLP-1-(7-37)amide; N-epsilon37-[2-(2-{2-[2-(2-{2-[(S)-4-carboxy-4-(19-carboxynonadecanoylamino) butyrylamino] ethoxy}ethoxy) acetylamino]ethoxy} ethoxy)acetyl] [DesaminoHis7,Glu22,Arg26, Arg34,Lys37]GLP-1-(7-37); N-epsilon37-(2-{2-[2-((S)-4-carboxy-4-{(S)-4-carboxy-4-[(S)-4-carboxy-4-(19-carboxy-nonadecanoylamino)butyrylamino]butyrylamino}butyrylamino)ethoxy]ethoxy}acetyl)[DesaminoHis7,Glu22,Arg26,Arg34,Lys37]GLP-1-(7-37); N-epsilon37-{2-[2-(2-{(S)-4-[(S)-4-(12-{4-[16-(2-tert-Butyl-2H-tetrazol-5-yl)-hexadecanoylsulfamoyl] butyrylamino}dodecanoylamino)-4-carboxybutyrylamino]-4-carboxybutyrylamino} ethoxy)ethoxy]acetyl}[DesaminoHis7,Glu22,Arg26,Arg34,Lys37]GLP-1 (7-37); N-epsilon37-[2-(2-{2-[2-(2-{2-[(S)-4-carboxy-4-(17-carboxy-heptadecanoylamino)-butyrylamino]-ethoxy}-ethoxy)-acetylamino]-ethoxy}-ethoxy)-acetyl][Aib8,Glu22, Arg26,Arg34,Lys37]GLP-1-(7-37); N-alpha37-[2-(2-{2-[2-(2-{2-[(S)-4-carboxy-4-(17-carboxy-heptadecanoylamino)-butyrylamino]-ethoxy}-ethoxy)-acetylamino]-ethoxy}-ethoxy)-acetyl] [Aib8,Glu22,Arg26,Arg34,epsilon-Lys37]GLP-1-(7-37)peptide; N-epsilon37-[2-(2-{2-[2-(2-{2-[(S)-4-carboxy-4-(17-carboxy-heptadecanoylamino)-butyrylamino]-ethoxy}-ethoxy)-acetylamino]-ethoxy}-ethoxy)-acetyl] [desaminoHis7, Glu22,Arg26,Arg34,Lys37] GLP-1-(7-37); N-epsilon36-[2-(2-{2-[2-(2-{2-[(S)-4-carboxy-4-(15-carboxy-pentadecanoylamino)-butyrylamino]-ethoxy}-ethoxy)-acetylamino]-ethoxy}-ethoxy)-acetyl] [desaminoHis7, Glu22,Arg26,Glu30,Arg34,Lys36]GLP-1-(7-37)-Glu-Lys peptide; N-epsilon37-[2-(2-{2-[2-(2-{2-[(S)-4-carboxy-4-({trans-4-[(19-carboxynonadecanoylamino)methyl]cyclohexanecarbonyl}amino)butyrylamino]ethoxy}etho xy)acetylamino]ethoxy}ethoxy)acetyl] [Aib8,Glu22,Arg26,Arg34,Lys37]GLP-1-(7-37); N-epsilon37-[2-(2-{2-[2-(2-{2-[(S)-4-carboxy-4-(17-carboxyheptadecanoylamino)-butyrylamino]-ethoxy}-ethoxy)-acetylamino]-ethoxy}-ethoxy)-acetyl]-[Aib8,Glu22, Arg26,Arg34,Aib35,Lys37]GLP-1-(7-37); N-epsilon37-[(S)-4-carboxy-4-(2-{2-[2-(2-{2-[2-(17-carboxyheptadecanoylamino) ethoxy] ethoxy} acetylamino) ethoxy] ethoxy}acetylamino) butyryl] [Aib8,Glu22,Arg26,34,Lys37] GLP-1 (7-37); N-epsilon37-[2-(2-[2-(2-[2-(2-[4-(17-carboxyheptadecanoylamino)-4(S)-carboxybutyrylamino]ethoxy)ethoxy]acetylamino)ethoxy]ethoxy)acetyl] [ImPr7,Glu22, Arg26,34,Lys37], GLP-1-(7-37); N-epsilon26-{2-[2-(2-{2-[2-(2-{(S)-4-carboxy-4-[10-(4-carboxyphenoxy)decanoylamino]butyrylamino}ethoxy)ethoxy] acetylamino}ethoxy) ethoxy]acetyl}, N-epsilon37-{2-[2-(2-{2-[2-(2-{(S)-4-carboxy-4-[10-(4-carboxyphenoxy) decanoylamino] butyrylamino}ethoxy)ethoxy]acetylamino}ethoxy) ethoxy]acetyl}-[Aib8,Arg34,Lys37]GLP-1(7-37)-OH; N-epsilon26 (17-carboxyheptadecanoyl)-[Aib8,Arg34]GLP-1-(7-37)-peptide; N-epsilon26-(19-carboxynonadecanoyl)-[Aib8,Arg34]GLP-1-(7-37); N-epsilon26-(4-{[N-(2-carboxyethyl)-N-(15-carboxypentadecanoyl)amino]methyl}benzoyl[Arg34]GLP-1-(7-37); N-epsilon26-[2-(2-[2-(2-[2-(2-[4-(17-carboxyheptadecanoylamino)-4(S)-carboxybutyrylamino]ethoxy)ethoxy]acetylamino) ethoxy]ethoxy)acetyl][Aib8,Arg34]GLP-1-(7-37); N-epsilon26-[2-(2-[2-(2-[2-(2-[4-(19-carboxynonadecanoylamino)-4(S)-carboxybutyrylamino]ethoxy)ethoxy]acetylamino)ethoxy]ethoxy)acetyl] [Aib8,Arg34]GLP-1-(7-37); N-epsilon26-[2-(2-[2-(2-[2-(2-[4-(17-carboxyheptadecanoylamino)-4(S)-carboxybutyrylamino]ethoxy)ethoxy]acetylamino)ethoxy]ethoxy)acetyl] [3-(4-Imidazolyl)Propionyl7,Arg34]GLP-1-(7-37); N-epsilon26-[2-(2-[2-(2-[2-(2-[4-(17-carboxyheptadecanoylamino)-(carboxymethyl-amino)acetylamino]ethoxy)ethoxy]acetylamino)ethoxy]ethoxy)acetyl] [Aib8,Arg34]GLP-1-(7-37); N-epsilon26-[2-(2-[2-(2-[2-(2-[4-(17-carboxyheptadecanoylamino)-3(S)-Sulfopropionylamino]ethoxy)ethoxy]acetylamino)ethoxy]ethoxy)acetyl] [Aib8,Arg34]GLP-1-(7-37); N-epsilon26-[2-(2-[2-(2-[2-(2-[4-(17-carboxyheptadecanoylamino)-4(S)-carboxybutyrylamino]ethoxy)ethoxy]acetylamino)ethoxy]ethoxy)acetyl][Gly8,Arg34]GLP-1-(7-37); N-epsilon26-[2-(2-[2-(2-[2-(2-[4-(17-carboxyheptadecanoylamino)-4(S)-carboxybutyrylamino]ethoxy)ethoxy]acetylamino)ethoxy]ethoxy)acetyl] [Aib8,Arg34]GLP-1-(7-37)-amide; N-epsilon26-[2-(2-[2-(2-[2-(2-[4-(17-carboxyheptadecanoylamino)-4(S)-carboxybutyrylamino]ethoxy)ethoxy]acetylamino)ethoxy]ethoxy)acetyl][Aib8,Arg34,Pro37]GLP-1-(7-37)amide; Aib8,Lys26(N-epsilon26-{2-(2-(2-(2-[2-(2-(4-(pentadecanoylamino)-4-carboxybutyrylamino)ethoxy)ethoxy]acetyl)ethoxy) ethoxy)acetyl)}), Arg34)GLP-1-(7-37)-OH; N-epsilon26-[2-(2-[2-(2-[2-(2-[4-{[N-(2-carboxyethyl)-N-(17-carboxyheptadecanoyl)amino]methyl}benzoyl)amino]ethoxy) ethoxy]acetylamino)ethoxy]ethoxy)acetyl] [Aib8,Arg34]GLP-1(7-37); N-alpha7-formyl, N-epsilon26-[2-(2-[2-(2-[2-(2-[4-(17-carboxyheptadecanoyl-amino)-4(S)-carboxy-butyrylamino]ethoxy)ethoxy]acetylamino)ethoxy]ethoxy)acetyl] [Arg34]GLP-1-(7-37); N-epsilon2626-[2-(2-[2-(2-[2-(2-[4-(17-carboxyheptadecanoylamino)-4(S)-carboxy-butyrylamino]ethoxy)ethoxy]acetylamino)ethoxy]ethoxy)acetyl] [Aib8, Glu22, Arg34] GLP-1-(7-37); N-epsilon26{3-[2-(2-{2-[2-(2-{2-[2-(2-[4-(15-(N—((S)-1,3-dicarboxypropyl) carbamoyl)pentadecanoylamino)-(S)-4-carboxybutyrylamino] ethoxy)ethoxy]ethoxy}ethoxy)ethoxy]ethoxy}ethoxy)ethoxy]propionyl} [Aib8,Arg34]GLP-1-(7-37); N-epsilon26-[2-(2-[2-(2-[2-(2-[4-{[N-(2-carboxyethyl)-N-(17-carboxy-heptadecanoyl)amino]methyl}benzoyl)amino](4(S)-carboxybutyryl-amino)ethoxy) ethoxy]acetylamino)ethoxy]ethoxy)acetyl] [Aib8,Arg34] GLP-1(7-37); N-epsilon26-{(S)-4-carboxy-4-((S)-4-carboxy-4-((S)-4-carboxy-4-((S)-4-carboxy-4-(19-carboxy-nonadecanoylamino)butyrylamino)butyrylamino)butyrylamino) butyrylamino}[Aib8,Arg34]GLP-1-(7-37); N-epsilon26-4-(17-carboxyheptadecanoyl-amino)-4(S)-carboxybutyryl-[Aib8,Arg34]GLP-1-(7-37); N-epsilon26-{3-[2-(2-{2-[2-(2-{2-[2-(2-[4-(17-carboxyheptadecanoylamino)-4(S)-carboxybutyrylamino]ethoxy)ethoxy]ethoxy}ethoxy)ethoxy]ethoxy}ethoxy)ethoxy]propiony 1[Aib8,Arg34]GLP-1-(7-37); N-epsilon26-12-(2-(2-(2-[2-(2-(4-(17-carboxyheptadecanoylamino)-4-carboxybutyrylamino) ethoxy)ethoxy]acetyl)ethoxy)ethoxy)acetyl)}-[Aib8,22,27,30,35,Arg34,Pro37, Lys26]GLP-1 (7-37)amide; N-epsilon26-[2-(2-[2-[4-(21-carboxyuneicosanoylamino)-4(S)-carboxybutyrylamino]ethoxy]ethoxy)acetyl] [Aib8,Arg34]GLP-1-(7-37); and N-epsilon26-[2-(2-[2-(2-[2-(2-[4-(21-carboxyuneicosanoylamino)-4(S)-carboxybutyrylamino]ethoxy)ethoxy]acetylamino)ethoxy]ethoxy)acetyl] [Aib8,Arg34]GLP-1-(7-37).

The term solid dosage form can refer to, but not limited to, a tablet, or a capsule filled with solids, or a capsule filled with a solution.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated.

The term “therapeutically effective amount,” as used herein, refers to that amount of a therapeutic agent (e.g., empagliflozin and/or semaglutide) sufficient to result in amelioration of one or more symptoms of a disorder or condition (e.g., Type 2 Diabetes), or prevent appearance or advancement of a disorder or condition, or cause regression of or cure from the disorder or condition.

The term “subject” (alternatively referred to herein as “patient”) as used herein, refers to an animal, preferably a mammal, most preferably a human, who has been the object of treatment, observation or experiment.

### EXAMPLES

### Example 1. Formulations

This example shows that 1) SNAC and sodium caprate formulated with semaglutide enables oral absorption of semaglutide; 2) co-formulation of semaglutide and empagliflozin in the same tablet with SNAC and sodium caprate.

Jardiance (empagliflozin 10 mg tablet) was from Boehringer Ingelheim, with a batch number of 0058448. The inactive ingredients claimed in this product label include lactose monohydrate, microcrystalline cellulose, hydroxypropyl cellulose, croscarmellose sodium, colloidal silicon dioxide and magnesium stearate. This product was used to evaluated the PK absorption of empagliflozin without combination of semaglutide.

Appropriate amounts of drug (e.g., semaglutide sodium) and excipients, such as SNAC or SNAC and sodium caprate (“C10”), and other optional excipients if present, were mixed in water to form a solution. The solution was then freeze dried for next step. Then empagliflozin was blended with the lyophilized mixer to provide the formulation.

Representative procedure for preparing formulation containing semaglutide, empagliflozin, SNAC, and C10: Weigh semaglutide sodium and add it into a glass vial. Add ˜15 ml of water to dissolve each 65 mg of semaglutide sodium. Then weigh SNAC and add it into above semaglutide water solution. Use a stir bar for stirring. Next add sodium caprate (and other excipients, if necessary), into the solution and stir the solution to make all excipients dissolved. The solution was then freeze-dried. Next the lyophilized mixer was blended with empagliflozin to form a uniform mixer.

Formulation 1 prepared according to this procedure contains 10 mg of semaglutide, 300 mg SNAC and 150 mg sodium caprate. Formulation 2 prepared according to this procedure contains 10 mg of semaglutide, 10 mg empagliflozin, 300 mg SNAC and 150 mg sodium caprate. Jardiance was used as a control group, which contains empagliflozin but without semaglutide, SNAC or sodium caprate.

The freeze-dried powder was then processed into a tablet form, which was used for the examples herein. The tablet formation was conducted using a manual compressor, a round tablet mold with a diameter of 12 mm, and an arc in the surface of the mold.

### Example 2. Pharmacokinetics Studies

This example describes pharmacokinetics (“PK”) studies of oral semaglutide 10 mg in a tablet, commercial tablet Jardiance 10 mg, and combination of semaglutide and empagliflozin in the same tablet.

Dogs were raised in separated cages. Before an experiment, two meals were supplied to the dogs at about 3 p.m. and 7 a.m. every day. The foods are fodder with water in a bowl. In the day before the experiment, after feeding the dogs at 3 p.m., the dogs were fasted overnight for about 18 h. The dogs have access to water overnight, but water was removed at 1 h before dosing. Before dosing, the dogs were put into a sling and then a 0.8 ml of blood was collected from each dog's leg vein using a sterile disposable syringe.

Tablet oral dosing: open the cages and let the dogs climb on the cage by their forelegs. Catch their mouths and open their mouths by hands. Workers will keep a dog's mouth open and put the tablet into the dog's throat using their fingers. Then close the dog's mouth and keep the mouth closed for about ten seconds. The dog would swallow the tablet naturally without any water.

After dosing, 0.8 ml of blood samples were collected into EDTA anticoagulant tubes at the following time points: 15 min, 30 min, 1 h, 2 h, 4 h, 6 h, 8 h, 24 h, 48 h, 72 h, 96 h, 120 h and 168 h from the dog's foreleg vein. After turning upside down for several times for mixing, the blood samples were centrifuged at 3500 rpm for ten minutes. The supernatant plasma samples were removed to clean the tubes and stored at −20° C. in the freezer before measurements. After the collection of the 8 h time point samples, the dogs were released, and food and water were provided to them immediately.

A LC-MS method with MRM mode is used to measure the plasma concentration of semaglutide. 150 μl of acetonitrile with internal standard was added into 50 μl of plasma to precipitate the proteins. After vortexing for 30 seconds, the samples were put into sonication for two minutes. The samples were centrifuged at 15400 G for 10 minutes. The supernatant was then removed for analysis. The mass spectrum is AB (Q TRAP 6500/LC-20A), SHIMADZU. A Sepax Bio-C18(4.6*150 mm, 5 m) was used. The parameters of the LC-MS are summarized below:
Mobile phase: A: H2O (0.1% Formic acid and 5 mM ammonium acetate; B: ACN (0.2% Formic acid)
Flow rate: 1 ml/min
Column temperature: 40° C.
CAD(psi): high
Gas1(psi): 60
IS(V): 5500
CUR(psi): 45
Gas2(psi): 55
TEM(° C.):600
Mode: ESI/+
Semaglutide ion pairs: 1029.2 m/z˜136.1 m/z, collision energy is 100V.

A LC-MS method with MRM mode is used to measure the plasma concentration of empagliflozin. 150 μl of acetonitrile with internal standard was added into 50 μl of plasma to precipitate the proteins. After vortexing for 30 seconds, the samples were put into sonication for two minutes. The samples were centrifuged at 15400 G for 10 minutes. The supernatant was then removed for analysis. The mass spectrum is AB (Q TRAP 6500/LC-20A), SHIMADZU. A Zorbax Eclipse Plus C18 (4.6*150 mm, 5 m) was used. The parameters of the LC-MS are summarized below:
Mobile phase: A: H2O (0.05% acetic acid; B: ACN
Flow rate: 0.7 ml/min
Column temperature: 40° C.
CAD(psi): medium
Gas1(psi): 55
IS(V): −4500
CUR(psi): 35
Gas2(psi): 55
TEM(° C.):550
Mode: ESI/−

Semaglutide Ion Pairs: 1029.2 m/z·136.1 m/z, Collision Energy is 100V.

### Experimental Results

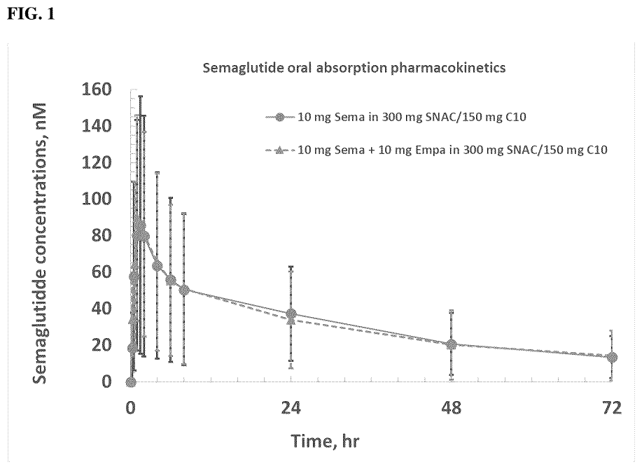

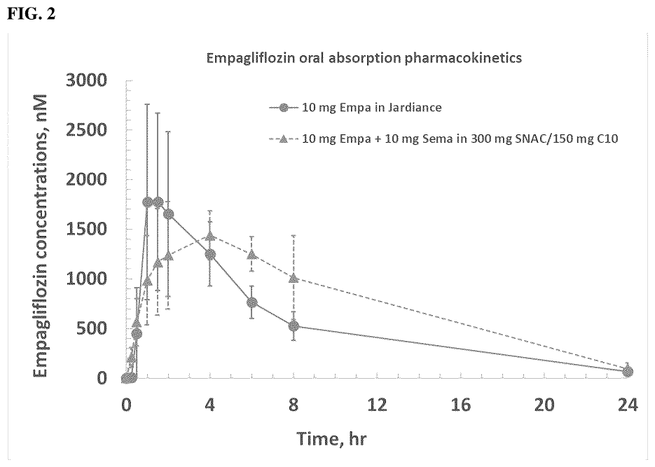

Experimental results in beagle dogs are summarized for the pharmacokinetic parameters of semaglutide and empagliflozin in Tables 1 and 2. The concentration-time profiles of semaglutide and empagliflozin are shown in FIGS. 1 and 2.

The area under the concentration-time (AUC) of semaglutide was calculated using the linear trapezoidal rule. The inter-individual variability was assessed with the coefficient of variation (CV), calculated by the standard deviation divided by the mean.

TABLE 1
Summary of Pharmacokinetic Results of Empagliflozin
in the tablets with or without Semaglutide (Sema)

| | Tmax | Cmax | | AUClast | |
|---|---|---|---|---|---|
| Formulations | hr | nM | CV_Cmax | nM*hr | CV_AUC |
| Without Sema | 1.5 | 1776 | 21 | 13302 | 14 |
| With Sema | 4.0 | 1674 | 10 | 17977 | 19 |

TABLE 2
Summary of Pharmacokinetic Results of Semaglutide
in the tablets with or without Empagliflozin (Empa)

| | Tmax | Cmax | | AUClast | |
|---|---|---|---|---|---|
| Formulations | hr | nM | CV_Cmax | nM*hr | CV_AUC |
| Without Empa | 1.0 | 89.9 | 63 | 2242 | 79 |
| With Empa | 1.5 | 86.7 | 82 | 2194 | 71 |

Note, the difference in Tmax between two formulations was due to the actual sampling time difference; that is, one was taken at 1 hr and the other was taken at 1.5 hr.

In comparison to the formulation containing semaglutide alone, the co-formulation of semaglutide with empagliflozin showed almost no impact on the pharmacokinetics of semaglutide, in terms of AUC, Cmax or even time-course profiles of semaglutide.

In comparison to the formulation containing empagliflozin alone in the commercial product Jardiance, the co-formulation of empagliflozin with semaglutide, unexpectedly discovered that, 1) the AUC of empagliflozin was enhanced by about 35%; 2) the Cmax was decreased as well; 3) the pharmacokinetic profile was much prolonged. The increased AUC and prolonged exposure may result in better pharmacological effect, while lowering the peak Cmax of empagliflozin may potentially decrease its side effects.

Overall, the co-formulation of semaglutide and empagliflozin in this invention, two distinct molecular entities with different physic-chemical, biological and metabolic properties, not only demonstrated no negative interference between each other, but also showed superior pharmacokinetic profiles of empagliflozin. Therefore, such combination of drugs in a single tablet is expected to result in synergistic pharmacological and medical benefits while bringing in significant advantages in use convenience and compliance.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

With respect to aspects of the invention described as a genus, all individual species are individually considered separate aspects of the invention. If aspects of the invention are described as “comprising” a feature, embodiments also are contemplated “consisting of or “consisting essentially of” the feature.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. To the extent that any meaning or definition of a term in this document conflicts with any meaning or definition of the same term in a document incorporated by reference, the meaning or definition assigned to that term in this document shall govern.

1. A pharmaceutical composition comprising:
(a) an SGLT-2 inhibitor; and
one or more ingredients selected from (b) an aliphatic acid of Formula I: RCOOH, wherein R represents an aliphatic group, or a pharmaceutically acceptable salt thereof; and (c) a compound of Formula II: 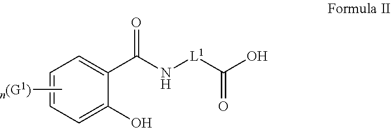
or a pharmaceutically acceptable salt thereof, wherein:
n is an integer selected from 0, 1, 2, 3, or 4;
G¹ at each occurrence is independently OH, NH₂, NH(C₁₋₄ alkyl), N(C₁₋₄ alkyl)(C₁₋₄ alkyl), halogen (e.g., Cl), C₁₋₄ alkyl, or C₁₋₄ alkoxy (e.g., OCH₃); and
L¹ is a substituted or unsubstituted C₂-C₁₆ alkylene, or substituted or unsubstituted C₂-C₁₆ alkenylene.

2. The pharmaceutical composition of claim 1, comprising (a) the SGLT-2 inhibitor; and (b) the aliphatic acid of Formula I, or a pharmaceutically acceptable salt thereof.

3. The pharmaceutical composition of claim 1, comprising (a) the SGLT-2 inhibitor; and (c) the compound of Formula II, or a pharmaceutically acceptable salt thereof.

4. The pharmaceutical composition of claim 1, comprising (a) the SGLT-2 inhibitor, (b) the aliphatic acid of Formula I, or a pharmaceutically acceptable salt thereof; and (c) the compound of Formula II, or a pharmaceutically acceptable salt thereof.

5. The pharmaceutical composition of claim 1, formulated for oral administration.

6. The pharmaceutical composition of claim 1, which upon oral administration to a human subject in need thereof, delivers a therapeutically effective amount of the SGLT-2 inhibitor to the human subject.

7. The pharmaceutical composition of claim 1, wherein when the aliphatic acid of Formula I or a pharmaceutically acceptable salt thereof is present, R in Formula I represents an alkyl group having 1-30 carbon atoms, e.g., R is —(CH₂)₁₋₁₈CH₃.

8. (canceled)

9. (canceled)

10. The pharmaceutical composition of claim 1, wherein when the aliphatic acid of Formula I or a pharmaceutically acceptable salt thereof is present, the aliphatic acid of Formula I is a linear aliphatic acid having 2 to 20 carbon atoms, such as caprylic acid, capric acid, or lauric acid.

11. (canceled)

12. (canceled)

13. (canceled)

14. The pharmaceutical composition of claim 1, wherein when the compound of Formula II or a pharmaceutically acceptable salt thereof is present, in Formula II, L¹ is a substituted or unsubstituted C₂-C₁₆ alkylene.

15. (canceled)

16. (canceled)

17. (canceled)

18. The pharmaceutical composition of claim 1, wherein when the compound of Formula II or a pharmaceutically acceptable salt thereof is present, the compound of Formula II is

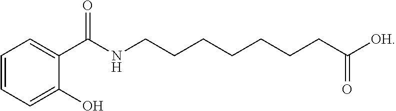 [SMILES: O=C(O)CCCCCCCNC(=O)c1ccccc1O]

19. The pharmaceutical composition of claim 1, wherein when the compound of Formula II or a pharmaceutically acceptable salt thereof is present, the compound of Formula II is

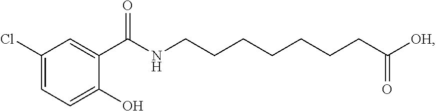 [SMILES: O=C(O)CCCCCCCNC(=O)c1cc(Cl)ccc1O]

20. (canceled)

21. The pharmaceutical composition of claim 1, comprising a sodium salt of

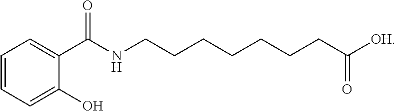 [SMILES: O=C(O)CCCCCCCNC(=O)c1ccccc1O]

22. The pharmaceutical composition of claim 21, comprising the aliphatic acid of Formula I or a pharmaceutically acceptable salt thereof is present, wherein the aliphatic acid of Formula I is capric acid.

23. The pharmaceutical composition of claim 1, wherein as applicable, the weight ratio of (b) the aliphatic acid of Formula I or pharmaceutically acceptable salt thereof to (c) the compound of Formula II or pharmaceutically acceptable salt thereof, (b)/(c), ranges from about 20:1 to about 1:20, such as 5:1 to 1:5, e.g., about 1:2.

24. (canceled)

25. (canceled)

26. The pharmaceutical composition of claim 1, further comprising an incretin.

27. (canceled)

28. (canceled)

29. (canceled)

30. The pharmaceutical composition of claim 26, wherein the incretin is semaglutide, liraglutide, dulaglutide, lixisenatide, exenatide, or a combination thereof.

31. The pharmaceutical composition of claim 1, wherein the SGLT-2 inhibitor is empagliflozin, canagliflozin, dapagliflozin, ertugliflozin, or a combination thereof.

32. The pharmaceutical composition of claim 26, comprising the incretin and the SGLT-2 inhibitor, wherein the SGLT-2 inhibitor is empagliflozin and the incretin is semaglutide.

33. (canceled)

34. (canceled)

35. (canceled)

36. (canceled)

37. A method of treating a disease or disorder, e.g., type-2 diabetes, obesity, NASH, or renal disease such as CKD, in a subject in need thereof, the method comprising orally administering the pharmaceutical composition of claim 1 to deliver a therapeutically effective amount of the SGLT-2 inhibitor and optionally also incretin to the subject.

38. A method of preparing the pharmaceutical composition of claim 26, as applicable, the method comprising (a) mixing the incretin with the compound of Formula II or pharmaceutically acceptable salt thereof, and optionally the aliphatic acid of Formula I or pharmaceutically acceptable salt thereof to form a mixture; (b) freeze-drying the mixture formed in (a) to form a freeze-dried mixture; and optionally (c) mixing the freeze-dried mixture with a pharmaceutically acceptable excipient.

39. (canceled)

40. (canceled)

41. (canceled)

42. (canceled)

43. (canceled)

44. (canceled)

45. (canceled)

46. (canceled)

47. (canceled)


SEQUENCE LISTING (3 sequences)

SEQ ID NO: 1 - protein, 31 aa, Homo sapiens
HAEGTFTSDVSSYLEGQAAKEFIAWLVKGRG

SEQ ID NO: 2 - protein, 40 aa, Homo sapiens
HGEGTFITSDLSKQMEEEAVRLFIEWLKNGGPSSGAPPPS

SEQ ID NO: 3 - protein, 31 aa, synthetic construct
  SITE 2: note = X is Aib (2-Aminoisobutyric acid)
  MOD_RES 20: note = Lysine, N-epsilon-[2-(2-{2-[2-(2-{2-[(S)-4-Carboxy- 4-(17-carboxyheptadecanoylamino)butyryl-amino]ethoxy}ethox y)acetylamino]ethoxy}ethoxy)acetyl]
HXEGTFTSDVSSYLEGQAAKEFIAWLVRGRG